(19) United States
(12) Patent Application Publication
(10) Pub. No.: US 2012/0302954 A1
(43) Pub. Date: Nov. 29, 2012

(54) EXPANDABLE DEVICES COATED WITH A PACLITAXEL COMPOSITION

(76) Inventor: Jonathon Z. Zhao, Belle Mead, NJ (US)

(21) Appl. No.: 13/115,378

(22) Filed: May 25, 2011

(51) Int. Cl. A61M 29/00

(52) U.S. Cl. 604/103.02

(57) ABSTRACT

Medical devices may be utilized for local and regional therapeutic agent delivery. These therapeutic agents or compounds may reduce a biological organism's reaction to the introduction of the medical device to the organism. In addition, these therapeutic drugs, agents and/or compounds may be utilized to promote healing, including the prevention of thrombosis. The drugs, agents, and/or compounds may also be utilized to treat specific disorders, including restenosis, vulnerable plaque, and atherosclerosis in type 2 diabetic patients.

### BACKGROUND OF THE INVENTION

1. Field of the Invention

The present invention relates to the local and/or regional administration of therapeutic agents and/or therapeutic agent combinations, and more particularly to expandable medical devices for the local and/or regional delivery of therapeutic agents and/or therapeutic agent combinations for the prevention and treatment of vascular disease.

2. Discussion of the Related Art

Many individuals suffer from circulatory disease caused by a progressive blockage of the blood vessels that perfuse the heart and other major organs. More severe blockage of blood vessels in such individuals often leads to hypertension, ischemic injury, stroke, or myocardial infarction. Atherosclerotic lesions, which limit or obstruct coronary blood flow, are the major cause of ischemic heart disease. Percutaneous transluminal coronary angioplasty is a medical procedure whose purpose is to increase blood flow through an artery. Percutaneous transluminal coronary angioplasty is the predominant treatment for coronary vessel stenosis. The increasing use of this procedure is attributable to its relatively high success rate and its minimal invasiveness compared with coronary bypass surgery. A limitation associated with percutaneous transluminal coronary angioplasty is the abrupt closure of the vessel, which may occur immediately after the procedure and restenosis, which occurs gradually following the procedure. Additionally, restenosis is a chronic problem in patients who have undergone saphenous vein bypass grafting. The mechanism of acute occlusion appears to involve several factors and may result from vascular recoil with resultant closure of the artery and/or deposition of blood platelets and fibrin along the damaged length of the newly opened blood vessel.

Restenosis after percutaneous transluminal coronary angioplasty is a more gradual process initiated by vascular injury. Multiple processes, including thrombosis, inflammation, growth factor and cytokine release, cell proliferation, cell migration and extracellular matrix synthesis each contribute to the restenotic process.

Upon pressure expansion of an intracoronary balloon catheter during angioplasty and/or stent implantation, smooth muscle cells and endothelial cells within the vessel wall become injured, initiating a thrombotic and inflammatory response. Cell derived growth factors such as platelet derived growth factor, basic fibroblast growth factor, epidermal growth factor, thrombin, etc., released from platelets, invading macrophages and/or leukocytes, or directly from the smooth muscle cells provoke a proliferative and migratory response in medial smooth muscle cells. These cells undergo a change from a contractile phenotype to a synthetic phenotype characterized by only a few contractile filament bundles, extensive rough endoplasmic reticulum, Golgi and free ribosomes. Proliferation/migration usually begins within one to two days' post-injury and peaks several days thereafter (Campbell and Campbell, 1987; Clowes and Schwartz, 1985).

Daughter cells migrate to the intimal layer of arterial smooth muscle and continue to proliferate and secrete significant amounts of extracellular matrix proteins. Proliferation, migration and extracellular matrix synthesis continue until the damaged endothelial layer is repaired at which time proliferation slows within the intima, usually within seven to fourteen days post-injury. The newly formed tissue is called neointima. The further vascular narrowing that occurs over the next three to six months is due primarily to negative or constrictive remodeling.

Simultaneous with local proliferation and migration, inflammatory cells adhere to the site of vascular injury. Within three to seven days post-injury, inflammatory cells have migrated to the deeper layers of the vessel wall. In animal models employing either balloon injury or stent implantation, inflammatory cells may persist at the site of vascular injury for at least thirty days (Tanaka et al., 1993; Edelman et al., 1998). Inflammatory cells therefore are present and may contribute to both the acute and chronic phases of restenosis.

Unlike systemic pharmacologic therapy, stents have proven useful in significantly reducing restenosis. Typically, stents are balloon-expandable slotted metal tubes (usually, but not limited to, stainless steel), which, when expanded within the lumen of an angioplastied coronary artery, provide structural support through rigid scaffolding to the arterial wall. This support is helpful in maintaining vessel lumen patency. In two randomized clinical trials, stents increased angiographic success after percutaneous transluminal coronary angioplasty, by increasing minimal lumen diameter and reducing, but not eliminating, the incidence of restenosis at six months (Serruys et al., 1994; Fischman et al., 1994).

Additionally, the heparin coating of stents appears to have the added benefit of producing a reduction in sub-acute thrombosis after stent implantation (Serruys et al., 1996). Thus, sustained mechanical expansion of a stenosed coronary artery with a stent has been shown to provide some measure of restenosis prevention, and the coating of stents with heparin has demonstrated both the feasibility and the clinical usefulness of delivering drugs locally, at the site of injured tissue. However, in certain circumstances it may not be desirable to leave any type of implantable device in the body.

Accordingly, there exists a need for drug/drug combinations and associated local delivery devices for the prevention and treatment of vascular injury causing intimal thickening which is either biologically induced, for example, atherosclerosis, or mechanically induced, for example, through percutaneous transluminal coronary angioplasty.

### SUMMARY OF THE INVENTION

A device for the local and/or regional delivery of rapamycin and/or paclitaxel formulations in accordance with the present invention may be utilized to overcome the disadvantages set forth above.

Medical devices may be utilized for local and regional therapeutic agent delivery. These therapeutic agents or compounds may reduce a biological organism's reaction to the introduction of the medical device to the organism. In addition, these therapeutic drugs, agents and/or compounds may be utilized to promote healing, including the prevention of thrombosis. The drugs, agents, and/or compounds may also be utilized to treat specific disorders, including restenosis, vulnerable plaque, and atherosclerosis in type 2 diabetic patients.

The drugs, agents or compounds will vary depending upon the type of medical device, the reaction to the introduction of the medical device and/or the disease sought to be treated. The type of coating or vehicle utilized to immobilize the drugs, agents or compounds to the medical device may also vary depending on a number of factors, including the type of medical device, the type of drug, agent or compound and the rate of release thereof.

The present invention is directed to balloons or other inflatable or expandable devices that may be temporarily positioned within a body to deliver a therapeutic agent and/or continuation of therapeutic agents and then removed. The therapeutic agents may include various formulations of rapamycin and/or paclitaxel. This type of delivery device may be particularly advantageous in the vasculature where stents may not be suitable, for example, in the larger vessels of the peripheral vascular system.

In use, the balloon or other inflatable or expandable device may be coated with one or more liquid formulations of therapeutic agent(s) and delivered to a treatment site. The act of inflation or expansion would, force the therapeutic agents into the surrounding tissue. The device may be kept in position for a period of between ten seconds to about five minutes depending upon the location. If utilized in the heart, shorter durations are required relative to other areas such as the leg.

In accordance with a first aspect, the present invention is directed to a medical device. The medical device comprising an expandable member having a first diameter for insertion into a vessel and a second diameter for making contact with the vessel walls, and a non-aqueous formulation of a paclitaxel, including synthetic and semi-synthetic analogs thereof, affixed to and dried onto at least a portion of the surface of the expandable member, the dried, non-aqueous formulation comprising a paclitaxel, in a therapeutic dosage in the range of up to ten micrograms per square millimeter of expandable member surface area, an antioxidant in an amount of up to 5 percent by weight relative to the amount of paclitaxel, a film forming agent in a pharmaceutically acceptable range of between 0.05 percent to about 20 percent by weight relative to the amount of paclitaxel and substantially no volatile, non-aqueous solvent.

In accordance with another aspect, the present invention is directed to a non-aqueous formulation of a paclitaxel, including synthetic and semi synthetic analogs thereof. The non-aqueous formulation comprising paclitaxel in a therapeutic dosage range, an antioxidant in an amount of up to 5 percent by weight relative to the amount of paclitaxel, and a film forming agent in a pharmaceutically acceptable range of between 0.05 percent to about 20 percent by weight in an amount relative to the paclitaxel.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a graphical representation of the results of a bioactivity study in accordance with the present invention.

FIGS. 2A and 2B illustrate a dip coating process of a PTCA balloon in a liquid formulation of a therapeutic agent in accordance with the present invention.

FIG. 3 is a diagrammatic illustration of a first process for coating a PTCA balloon in accordance with the present invention.

FIG. 4 is a diagrammatic illustration of a second process for coating a PTCA balloon in accordance with the present invention.

FIG. 5 is a diagrammatic illustration of a stent on a coated PTCA balloon in accordance with the present invention.

FIG. 6 is a graphical representation of 30 day late lumen loss.

FIG. 7 is a graphical representation of minimal lumen diameter at 30 day follow up.

FIG. 8 comprises a series of images of three dried coating solutions on glass slides in accordance with the present invention.

FIG. 9 comprises before and after images of a coating solution containing no PVP on a glass slide in accordance with the present invention.

FIG. 10 comprises before and after images of a coating solution containing one percent K30 on a glass slide in accordance with the present invention.

FIG. 11 comprises before and after images of a coating solution containing one percent K90 on a glass slide in accordance with the present invention.

FIG. 12 comprises a series of images of three dried coating solutions on balloon surfaces in accordance with the present invention.

FIG. 13 comprises a series of images of three dried coating solutions on balloon surfaces after water immersion and abrasion in accordance with the present invention.

### DETAILED DESCRIPTION OF THE PREFERRED EMBODIMENTS

The drug/drug combinations and delivery devices of the present invention may be utilized to effectively prevent and treat vascular disease, including vascular disease caused by injury. Various medical treatment devices utilized in the treatment of vascular disease may ultimately induce further complications. For example, balloon angioplasty is a procedure utilized to increase blood flow through an artery and is the predominant treatment for coronary vessel stenosis. However, the procedure typically causes a certain degree of damage to the vessel wall, thereby potentially exacerbating the problem at a point later in time. Although other procedures and diseases may cause similar injury, exemplary embodiments of the present invention will be described with respect to the treatment of restenosis and related complications.

While exemplary embodiments of the invention will be described with respect to the treatment of restenosis and related complications following percutaneous transluminal coronary angioplasty, it is important to note that the local delivery of drug/drug combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach. Devices which serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using this drug-device combination approach. Perivascular wraps may be particularly advantageous, alone or in combination with other medical devices. The perivascular wraps may supply additional drugs to a treatment site. Essentially, any type of medical device may be coated in some fashion with a drug or drug combination which enhances treatment over use of the singular use of the device or pharmaceutical agent.

In addition to various medical devices, the coatings on these devices may be used to deliver therapeutic and pharmaceutic agents including: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

Rapamycin is a macrocyclic triene antibiotic produced by Streptomyces hygroscopicus as disclosed in U.S. Pat. No. 3,929,992. It has been found that rapamycin among other things inhibits the proliferation of vascular smooth muscle cells in vivo. Accordingly, rapamycin may be utilized in treating intimal smooth muscle cell hyperplasia, restenosis, and vascular occlusion in a mammal, particularly following either biologically or mechanically mediated vascular injury, or under conditions that would predispose a mammal to suffering such a vascular injury. Rapamycin functions to inhibit smooth muscle cell proliferation and does not interfere with the re-endothelialization of the vessel walls.

Rapamycin reduces vascular hyperplasia by antagonizing smooth muscle proliferation in response to mitogenic signals that are released during an angioplasty induced injury. Inhibition of growth factor and cytokine mediated smooth muscle proliferation at the late G1 phase of the cell cycle is believed to be the dominant mechanism of action of rapamycin. However, rapamycin is also known to prevent T-cell proliferation and differentiation when administered systemically. This is the basis for its immunosuppressive activity and its ability to prevent graft rejection.

The molecular events that are responsible for the actions of rapamycin, a known anti-proliferative, which acts to reduce the magnitude and duration of neointimal hyperplasia, are still being elucidated. It is known, however, that rapamycin enters cells and binds to a high-affinity cytosolic protein called FKBP12. The complex of rapamycin and FKPB12 in turn binds to and inhibits a phosphoinositide (PI)-3 kinase called the “mammalian Target of Rapamycin” or TOR. TOR is a protein kinase that plays a key role in mediating the downstream signaling events associated with mitogenic growth factors and cytokines in smooth muscle cells and T lymphocytes. These events include phosphorylation of p27, phosphorylation of p70 s6 kinase and phosphorylation of 4BP-1, an important regulator of protein translation.

It is recognized that rapamycin reduces restenosis by inhibiting neointimal hyperplasia. However, there is evidence that rapamycin may also inhibit the other major component of restenosis, namely, negative remodeling. Remodeling is a process whose mechanism is not clearly understood but which results in shrinkage of the external elastic lamina and reduction in lumenal area over time, generally a period of approximately three to six months in humans.

Negative or constrictive vascular remodeling may be quantified angiographically as the percent diameter stenosis at the lesion site where there is no stent to obstruct the process. If late lumen loss is abolished in-lesion, it may be inferred that negative remodeling has been inhibited. Another method of determining the degree of remodeling involves measuring in-lesion external elastic lamina area using intravascular ultrasound (IVUS). Intravascular ultrasound is a technique that can image the external elastic lamina as well as the vascular lumen. Changes in the external elastic lamina proximal and distal to the stent from the post-procedural timepoint to four-month and twelve-month follow-ups are reflective of remodeling changes.

Evidence that rapamycin exerts an effect on remodeling comes from human implant studies with rapamycin coated stents showing a very low degree of restenosis in-lesion as well as in-stent. In-lesion parameters are usually measured approximately five millimeters on either side of the stent i.e. proximal and distal. Since the stent is not present to control remodeling in these zones which are still affected by balloon expansion, it may be inferred that rapamycin is preventing vascular remodeling.

The local delivery of drug/drug combinations from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-thrombotic drugs, agents or compounds.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

The data in Table 1 below illustrate that in-lesion percent diameter stenosis remains low in the rapamycin treated groups, even at twelve months. Accordingly, these results support the hypothesis that rapamycin reduces remodeling.

TABLE 1.0
Angiographic In-Lesion Percent Diameter Stenosis
(%, mean ± SD and “n =”) In Patients Who Received a
Rapamycin-Coated Stent

| Coating | Post | 4-6 month | 12 month |
|---|---|---|---|
| Group | Placement | Follow Up | Follow Up |
| Brazil | 10.6 ± 5.7 (30) | 13.6 ± 8.6 (30) | 22.3 ± 7.2 (15) |
| Netherlands | 14.7 ± 8.8 | 22.4 ± 6.4 | — |

Additional evidence supporting a reduction in negative remodeling with rapamycin comes from intravascular ultrasound data that was obtained from a first-in-man clinical program as illustrated in Table 2 below.

TABLE 2.0
Matched IVUS data in Patients Who Received a Rapamycin-Coated Stent

| | | 4-Month | 12-Month |
|---|---|---|---|
| | | Follow-Up | Follow-Up |
| IVUS Parameter | Post (n =) | (n =) | (n =) |
| Mean proximal vessel area | 16.53 ± 3.53 | 16.31 ± 4.36 | 13.96 ± 2.26 |
| (mm²) | (27) | (28) | (13) |
| Mean distal vessel area | 13.12 ± 3.68 | 13.53 ± 4.17 | 12.49 ± 3.25 |
| (mm²) | (26) | (26) | (14) |

The data illustrated that there is minimal loss of vessel area proximally or distally which indicates that inhibition of negative remodeling has occurred in vessels treated with rapamycin-coated stents.

Other than the stent itself, there have been no effective solutions to the problem of vascular remodeling. Accordingly, rapamycin may represent a biological approach to controlling the vascular remodeling phenomenon.

It may be hypothesized that rapamycin acts to reduce negative remodeling in several ways. By specifically blocking the proliferation of fibroblasts in the vascular wall in response to injury, rapamycin may reduce the formation of vascular scar tissue. Rapamycin may also affect the translation of key proteins involved in collagen formation or metabolism.

Rapamycin may be delivered by a stent to control negative remodeling. Rapamycin may also be delivered systemically using an oral dosage form or a chronic injectable depot form or a patch to deliver rapamycin for a period ranging from about seven to forty-five days to achieve vascular tissue levels that are sufficient to inhibit negative remodeling. Such treatment is to be used to reduce or prevent restenosis when administered several days prior to elective angioplasty with or without a stent.

Data generated in porcine and rabbit models show that the release of rapamycin into the vascular wall from a nonerodible polymeric stent coating in a range of doses (35-430 ug/15-18 mm coronary stent) produces a peak fifty to fifty-five percent reduction in neointimal hyperplasia as set forth in Table 3 below. This reduction, which is maximal at about twenty-eight to thirty days, is typically not sustained in the range of ninety to one hundred eighty days in the porcine model as set forth in Table 4 below.

TABLE 3.0
Animal Studies with Rapamycin-coated stents.
Values are mean ± Standard Error of Mean

| | | | | | | Neointimal Area | % Change From | |
|---|---|---|---|---|---|---|---|---|
| Study | Duration | Stent¹ | Rapamycin | | N | (mm²) | Polyme | Metal |
| Porcine | | | | | | | | |
| 98009 | 14 days | Metal | | | 8 | 2.04 ± 0.17 | | |
| | | 1X + rapamycin | 153 | μg | 8 | 1.66 ± 0.17* | −42% | −19% |
| | | 1X + TC300 + rapamycin | 155 | μg | 8 | 1.51 ± 0.19* | −47% | −26% |
| 99005 | 28 days | Metal | | | 10 | 2.29 ± 0.21 | | |
| | | | | | 9 | 3.91 ± 0.60** | | |
| | | 1X + TC30 + rapamycin | 130 | μg | 8 | 2.81 ± 0.34 | | +23% |
| | | 1X + TC100 + rapamycin | 120 | μg | 9 | 2.62 ± 0.21 | | +14% |
| 99006 | 28 days | Metal | | | 12 | 4.57 ± 0.46 | | |
| | | EVA/BMA 3X | | | 12 | 5.02 ± 0.62 | | +10% |
| | | 1X + rapamycin | 125 | μg | 11 | 2.84 ± 0.31* ** | −43% | −38% |
| | | 3X + rapamycin | 430 | μg | 12 | 3.06 ± 0.17* ** | −39% | −33% |
| | | 3X + rapamycin | 157 | μg | 12 | 2.77 ± 0.41* ** | −45% | −39% |
| 99011 | 28 days | Metal | | | 11 | 3.09 ± 0.27 | | |
| | | | | | 11 | 4.52 ± 0.37 | | |
| | | 1X + rapamycin | 189 | μg | 14 | 3.05 ± 0.35 | | −1% |
| | | 3X + rapamycin/dex | 182/363 | μg | 14 | 2.72 ± 0.71 | | −12% |
| 99021 | 60 days | Metal | | | 12 | 2.14 ± 0.25 | | |
| | | 1X + rapamycin | 181 | μg | 12 | 2.95 ± 0.38 | | +38% |
| 99034 | 28 days | Metal | | | 8 | 5.24 ± 0.58 | | |
| | | 1X + rapamycin | 186 | μg | 8 | 2.47 ± 0.33** | | −53% |
| | | 3X + rapamycin/dex | 185/369 | μg | 6 | 2.42 ± 0.64** | | −54% |
| 20001 | 28 days | Metal | | | 6 | 1.81 ± 0.09 | | |
| | | 1X + rapamycin | 172 | μg | 5 | 1.66 ± 0.44 | | −8% |
| 20007 | 30 days | Metal | | | 9 | 2.94 ± 0.43 | | |
| | | 1XTC + rapamycin | 155 | μg | 10 | 1.40 ± 0.11* | | −52%* |
| Rabbit | | | | | | | | |
| 99019 | 28 days | Metal | | | 8 | 1.20 ± 0.07 | | |
| | | EVA/BMA 1X | | | 10 | 1.26 ± 0.16 | | +5% |
| | | 1X + rapamycin | 64 | μg | 9 | 0.92 ± 0.14 | −27% | −23% |
| | | 1X + rapamycin | 196 | μg | 10 | 0.66 ± 0.12* ** | −48% | −45% |
| 99020 | 28 days | Metal | | | 12 | 1.18 ± 0.10 | | |
| | | EVA/BMA 1X + rapamycin | 197 | μg | 8 | 0.81 ± 0.16 | | −32% |

¹Stent nomenclature: EVA/BMA 1X, 2X, and 3X signifies approx. 500 μg, 1000 μg, and 1500 μg total mass (polymer + drug), respectively. TC, top coat of 30 μg, 100 μg, or 300 μg drug-free BMA; Biphasic; 2 × 1X layers of rapamycin in EVA/BMA spearated by a 100 μg drug-free BMA layer.
²0.25 mg/kg/d × 14 d preceeded by a loading dose of 0.5 mg/kg/d × 3 d prior to stent implantation.
*p < 0.05 from EVA/BMA control.
**p < 0.05 from Metal;
^{#}Inflammation score: (0 = essentially no intimal involvement; 1 = <25% intima involved; 2 = ≧25% intima involved; 3 = >50% intima involved).

TABLE 4.0
180 day Porcine Study with Rapamycin-coated stents.
Values are mean ± Standard Error of Mean

| | | | | | Neointimal | % Change From | | Inflammation |
|---|---|---|---|---|---|---|---|---|
| Study | Duration | Stent¹ | Rapamycin | N | Area (mm²) | Polyme | Metal | Score # |
| 20007 | 3 days | Metal | | 10 | 0.38 ± 0.06 | | | 1.05 ± 0.06 |
| (ETP-2-002233-P) | | 1XTC + rapamycin | 155 μg | 10 | 0.29 ± 0.03 | | −24% | 1.08 ± 0.04 |
| | 30 days | Metal | | 9 | 2.94 ± 0.43 | | | 0.11 ± 0.08 |
| | | 1XTC + rapamycin | 155 μg | 10 | 1.40 ± 0.11* | | −52%* | 0.25 ± 0.10 |
| | 90 days | Metal | | 10 | 3.45 ± 0.34 | | | 0.20 ± 0.08 |
| | | 1XTC + rapamycin | 155 μg | 10 | 3.03 ± 0.29 | | −12% | 0.80 ± 0.23 |
| | | 1X + rapamycin | 171 μg | 10 | 2.86 ± 0.35 | | −17% | 0.60 ± 0.23 |
| | 180 days | Metal | | 10 | 3.65 ± 0.39 | | | 0.65 ± 0.21 |
| | | 1XTC + rapamycin | 155 μg | 10 | 3.34 ± 0.31 | | −8% | 1.50 ± 0.34 |
| | | 1X + rapamycin | 171 μg | 10 | 3.87 ± 0.28 | | +6% | 1.68 ± 0.37 |

The release of rapamycin into the vascular wall of a human from a nonerodible polymeric stent coating provides superior results with respect to the magnitude and duration of the reduction in neointimal hyperplasia within the stent as compared to the vascular walls of animals as set forth above.

Humans implanted with a rapamycin coated stent comprising rapamycin in the same dose range as studied in animal models using the same polymeric matrix, as described above, reveal a much more profound reduction in neointimal hyperplasia than observed in animal models, based on the magnitude and duration of reduction in neointima. The human clinical response to rapamycin reveals essentially total abolition of neointimal hyperplasia inside the stent using both angiographic and intravascular ultrasound measurements. These results are sustained for at least one year as set forth in Table 5 below.

TABLE 5.0
Patients Treated (N = 45 patients) with a Rapamycin-coated Stent

| | Sirolimus FIM | |
|---|---|---|
| | (N = 45 Patients, | 95% |
| Effectiveness Measures | 45 Lesions) | Confidence Limit |
| Procedure Success (QCA) | 100.0% (45/45) | [92.1%, 100.0%] |
| 4-month In-Stent Diameter | | |
| Stenosis (%) | | |
| Mean ± SD (N) | 4.8% ± 6.1% (30) | [2.6%, 7.0%] |
| Range (min, max) | (−8.2%, 14.9%) | |
| 6-month In-Stent Diameter | | |
| Stenosis (%) | | |
| Mean ± SD (N) | 8.9% ± 7.6% (13) | [4.8%, 13.0%] |
| Range (min, max) | (−2.9%, 20.4%) | |
| 12-month In-Stent Diameter | | |
| Stenosis (%) | | |
| Mean ± SD (N) | 8.9% ± 6.1% (15) | [5.8%, 12.0%] |
| Range (min, max) | (−3.0%, 22.0%) | |
| 4-month In-Stent Late Loss | | |
| (mm) | | |
| Mean ± SD (N) | 0.00 ± 0.29 (30) | [−0.10, 0.10] |
| Range (min, max) | (−0.51, 0.45) | |
| 6-month In-Stent Late Loss | | |
| (mm) | | |
| Mean ± SD (N) | 0.25 ± 0.27 (13) | [0.10, 0.39] |
| Range (min, max) | (−0.51, 0.91) | |
| 12-month In-Stent Late Loss | | |
| (mm) | | |
| Mean ± SD (N) | 0.11 ± 0.36 (15) | [−0.08, 0.29] |
| Range (min, max) | (−0.51, 0.82) | |
| 4-month Obstruction Volume | | |
| (%) (IVUS) | | |
| Mean ± SD (N) | 10.48% ± 2.78% (28) | [9.45%, 11.51%] |
| Range (min, max) | (4.60%, 16.35%) | |
| 6-month Obstruction Volume | | |
| (%) (IVUS) | | |
| Mean ± SD (N) | 7.22% ± 4.60% (13) | [4.72%, 9.72%], |
| Range (min, max) | (3.82%, 19.88%) | |
| 12-month Obstruction | | |
| Volume (%) (IVUS) | | |
| Mean ± SD (N) | 2.11% ± 5.28% (15) | [0.00%, 4.78%], |
| Range (min, max) | (0.00%, 19.89%) | |
| 6-month Target Lesion | 0.0% (0/30) | [0.0%, 9.5%] |
| Revascularization (TLR) | | |
| 12-month Target Lesion | 0.0% (0/15) | [0.0%, 18.1%] |
| Revascularization (TLR) | | |

QCA = Quantitative Coronary Angiography
SD = Standard Deviation
IVUS = Intravascular Ultrasound

Rapamycin produces an unexpected benefit in humans when delivered from a stent by causing a profound reduction in in-stent neointimal hyperplasia that is sustained for at least one year. The magnitude and duration of this benefit in humans is not predicted from animal model data.

These results may be due to a number of factors. For example, the greater effectiveness of rapamycin in humans is due to greater sensitivity of its mechanism(s) of action toward the pathophysiology of human vascular lesions compared to the pathophysiology of animal models of angioplasty. In addition, the combination of the dose applied to the stent and the polymer coating that controls the release of the drug is important in the effectiveness of the drug.

As stated above, rapamycin reduces vascular hyperplasia by antagonizing smooth muscle proliferation in response to mitogenic signals that are released during angioplasty injury. Also, it is known that rapamycin prevents T-cell proliferation and differentiation when administered systemically. It has also been determined that rapamycin exerts a local inflammatory effect in the vessel wall when administered from a stent in low doses for a sustained period of time (approximately two to six weeks). The local anti-inflammatory benefit is profound and unexpected. In combination with the smooth muscle anti-proliferative effect, this dual mode of action of rapamycin may be responsible for its exceptional efficacy.

Accordingly, rapamycin delivered from a local device platform, reduces neointimal hyperplasia by a combination of anti-inflammatory and smooth muscle anti-proliferative effects. Local device platforms include stent coatings, stent sheaths, grafts and local drug infusion catheters, porous or non-porous balloons or any other suitable means for the in situ or local delivery of drugs, agents or compounds. For example, as set forth subsequently, the local delivery of drugs, agents or compounds may be directly from a coating on a balloon.

The anti-inflammatory effect of rapamycin is evident in data from an experiment, illustrated in Table 6, in which rapamycin delivered from a stent was compared with dexamethasone delivered from a stent. Dexamethasone, a potent steroidal anti-inflammatory agent, was used as a reference standard. Although dexamethasone is able to reduce inflammation scores, rapamycin is far more effective than dexamethasone in reducing inflammation scores. In addition, rapamycin significantly reduces neointimal hyperplasia, unlike dexamethasone.

TABLE 6.0

| Group | | | | |
|---|---|---|---|---|
| Rapamycin | | Neointimal Area | % Area | Inflammation |
| (Rap) | N = | (mm²) | Stenosis | Score |
| Uncoated | 8 | 5.24 ± 1.65 | 54 ± 19 | 0.97 ± 1.00 |
| Dexamethasone | 8 | 4.31 ± 3.02 | 45 ± 31 | 0.39 ± 0.24 |
| (Dex) | | | | |
| Rapamycin | 7 | 2.47 ± 0.94* | 26 ± 10* | 0.13 ± 0.19* |
| (Rap) | | | | |
| Rap + Dex | 6 | 2.42 ± 1.58* | 26 ± 18* | 0.17 ± 0.30* |

*= significance level P < 0.05

Rapamycin has also been found to reduce cytokine levels in vascular tissue when delivered from a stent. The data illustrates that rapamycin is highly effective in reducing monocyte chemotactic protein (MCP-1) levels in the vascular wall. MCP-1 is an example of a proinflammatory/chemotactic cytokine that is elaborated during vessel injury. Reduction in MCP-1 illustrates the beneficial effect of rapamycin in reducing the expression of proinflammatory mediators and contributing to the anti-inflammatory effect of rapamycin delivered locally from a stent. It is recognized that vascular inflammation in response to injury is a major contributor to the development of neointimal hyperplasia.

Since rapamycin may be shown to inhibit local inflammatory events in the vessel it is believed that this could explain the unexpected superiority of rapamycin in inhibiting neointima.

As set forth above, rapamycin functions on a number of levels to produce such desired effects as the prevention of T-cell proliferation, the inhibition of negative remodeling, the reduction of inflammation, and the prevention of smooth muscle cell proliferation. While the exact mechanisms of these functions are not completely known, the mechanisms that have been identified may be expanded upon.

Studies with rapamycin suggest that the prevention of smooth muscle cell proliferation by blockade of the cell cycle is a valid strategy for reducing neointimal hyperplasia. Dramatic and sustained reductions in late lumen loss and neointimal plaque volume have been observed in patients receiving rapamycin delivered locally from a stent. Various embodiments of the present invention expand upon the mechanism of rapamycin to include additional approaches to inhibit the cell cycle and reduce neointimal hyperplasia without producing toxicity.

The cell cycle is a tightly controlled biochemical cascade of events that regulate the process of cell replication. When cells are stimulated by appropriate growth factors, they move from G₀ (quiescence) to the G1 phase of the cell cycle. Selective inhibition of the cell cycle in the G1 phase, prior to DNA replication (S phase), may offer therapeutic advantages of cell preservation and viability while retaining anti-proliferative efficacy when compared to therapeutics that act later in the cell cycle i.e. at S, G2 or M phase.

Accordingly, the prevention of intimal hyperplasia in blood vessels and other conduit vessels in the body may be achieved using cell cycle inhibitors that act selectively at the G1 phase of the cell cycle. These inhibitors of the G1 phase of the cell cycle may be small molecules, peptides, proteins, oligonucleotides or DNA sequences. More specifically, these drugs or agents include inhibitors of cyclin dependent kinases (cdk's) involved with the progression of the cell cycle through the G1 phase, in particular cdk2 and cdk4.

Examples of drugs, agents or compounds that act selectively at the G1 phase of the cell cycle include small molecules such as flavopiridol and its structural analogs that have been found to inhibit cell cycle in the late G1 phase by antagonism of cyclin dependent kinases. Therapeutic agents that elevate an endogenous kinase inhibitory protein^{kip} called P27, sometimes referred to as P27^{kip}, that selectively inhibits cyclin dependent kinases may be utilized. This includes small molecules, peptides and proteins that either block the degradation of P27 or enhance the cellular production of P27, including gene vectors that can transfact the gene to produce P27. Staurosporin and related small molecules that block the cell cycle by inhibiting protein kinases may be utilized. Protein kinase inhibitors, including the class of tyrphostins that selectively inhibit protein kinases to antagonize signal transduction in smooth muscle in response to a broad range of growth factors such as PDGF and FGF may also be utilized.

Any of the drugs, agents or compounds discussed herein may be administered either systemically, for example, orally, intravenously, intramuscularly, subcutaneously, nasally or intradermally, or locally, for example, stent coating, stent covering, local delivery catheter or balloon. In addition, the drugs or agents discussed above may be formulated for fast-release or slow release with the objective of maintaining the drugs or agents in contact with target tissues for a period ranging from three days to eight weeks.

As set forth above, the complex of rapamycin and FKPB12 binds to and inhibits a phosphoinositide (PI)-3 kinase called the mammalian Target of Rapamycin or TOR. An antagonist of the catalytic activity of TOR, functioning as either an active site inhibitor or as an allosteric modulator, i.e. an indirect inhibitor that allosterically modulates, would mimic the actions of rapamycin but bypass the requirement for FKBP12. The potential advantages of a direct inhibitor of TOR include better tissue penetration and better physical/chemical stability. In addition, other potential advantages include greater selectivity and specificity of action due to the specificity of an antagonist for one of multiple isoforms of TOR that may exist in different tissues, and a potentially different spectrum of downstream effects leading to greater drug efficacy and/or safety.

The inhibitor may be a small organic molecule (approximate mw<1000), which is either a synthetic or naturally derived product. Wortmanin may be an agent which inhibits the function of this class of proteins. It may also be a peptide or an oligonucleotide sequence. The inhibitor may be administered either sytemically (orally, intravenously, intramuscularly, subcutaneously, nasally, or intradermally) or locally (stent coating, stent covering, local drug delivery catheter). For example, the inhibitor may be released into the vascular wall of a human from a nonerodible polymeric stent coating. In addition, the inhibitor may be formulated for fast-release or slow release with the objective of maintaining the rapamycin or other drug, agent or compound in contact with target tissues for a period ranging from three days to eight weeks.

As stated previously, the implantation of a coronary stent in conjunction with balloon angioplasty is highly effective in treating acute vessel closure and may reduce the risk of restenosis. Intravascular ultrasound studies (Mintz et al., 1996) suggest that coronary stenting effectively prevents vessel constriction and that most of the late luminal loss after stent implantation is due to plaque growth, probably related to neointimal hyperplasia. The late luminal loss after coronary stenting is almost two times higher than that observed after conventional balloon angioplasty. Conventional balloon angioplasty is distinguished from drug delivery via balloons in that no drug is imparted by the balloon. Thus, inasmuch as stents prevent at least a portion of the restenosis process, the use of drugs, agents or compounds which prevent inflammation and proliferation, or prevent proliferation by multiple mechanisms, combined with a stent may provide the most efficacious treatment for post-angioplasty restenosis.

Further, insulin supplemented diabetic patients receiving rapamycin eluting vascular devices, such as stents, may exhibit a higher incidence of restenosis than their normal or non-insulin supplemented diabetic counterparts. Accordingly, combinations of drugs may be beneficial.

As used herein, rapamycin includes rapamycin and all analogs, derivatives and conjugates that bind to FKBP12, and other immunophilins and possesses the same pharmacologic properties as rapamycin including inhibition of TOR.

Although the anti-proliferative effects of rapamycin may be achieved through systemic use, superior results may be achieved through the local delivery of the compound. Essentially, rapamycin works in the tissues, which are in proximity to the compound, and has diminished effect as the distance from the delivery device increases. In order to take advantage of this effect, one would want the rapamycin in direct contact with the lumen walls.

As described herein, there are a number of advantages to the local or regional delivery of certain drugs, agents and/or compounds via means other than or in addition to delivery from an implantable medical device. However, the efficacy of the drugs, agents and/or compounds may, to a certain extent, depend on the formulation thereof. The mode of delivery may determine the formulation of the drug. Accordingly, different delivery devices may utilize different formulations. As illustrated above, drugs may be delivered from a stent; however, in other embodiments as described in detail subsequently, any number of devices may be utilized.

It is typically very difficult to create aqueous solution dosage forms of water insoluble and lipohilic (having an affinity for and/or tending to combine with lipids) drugs such as rapamycin and/or paclitaxel without resorting to substantial quantities of surfactants, co-solvents and the like. Often times, these excipients (inert substance that acts as a vehicle), such as Tween 20 and 80, Cremophor and polyethylene glycol (PEG) come with varying degrees of toxicity to the surrounding tissue. Accordingly, the use of organic co-solvents such as dimethol sulfoxide (DMSO), N-methylpyrrolidone (NMP) and ethanol need to be minimized to reduce the toxicity of the solvent. Essentially, the key for a liquid formulation of a water insoluble drug is to find a good combination of excipient and co-solvent, and an optimal range of the additives in the final dosage form to balance the improvement of drug solubility and necessary safety margins.

As the outstanding results from clinical trials of recent drug eluting stents such as the Cypher® and Taxus® drug eluting stents demonstrated, a prolonged local high concentration and tissue retention of a potent anti-inflammatory and anti-neoplastic agent released from a stent coating can substantially eliminate the neointimal growth following an angioplasty procedure. Rapamycin, released from the Cypher® stent has consistently demonstrated superior efficacy against restenosis after stent implantation as compared to a bare metal stent. However, there are clinical situations where a non-stent approach for the local delivery or regional delivery may be advantageous, including bifurcated junctions, small arteries and the restenosis of previously implanted stents. Accordingly, there may exist a need for potent therapeutics that only need to be deposited locally or regionally and the drug will exert its pharmacological functions mainly through its good lipophilic nature and long tissue retention property.

A locally or regionally delivered solution of a potent therapeutic agent, such as rapamycin, offers a number of advantages over a systemically delivered agent or an agent delivered via an implantable medical device. For example, a relatively high tissue concentration may be achieved by the direct deposition of the pharmaceutical agent in the arterial wall. Depending on the location of the deposition, a different drug concentration profile may be achieved than through that of a drug eluting stent. In addition, with a locally or regionally delivered solution, there is no need for a permanently implanted device such as a stent, thereby eliminating the potential side affects associated therewith, such as inflammatory reaction and long term tissue damage. It is, however, important to note that the locally or regionally delivered solution may be utilized in combination with drug eluting stents or other coated implantable medical devices. Another advantage of solution or liquid formulations lies in the fact that the adjustment of the excipients in the liquid formulation would readily change the drug distribution and retention profiles. In addition, the liquid formulation may be mixed immediately prior to the injection through a pre-packaged multi-chamber injection device to improve the storage and shelf life of the dosage forms.

A series of liquid formulations were developed for the local or regional delivery of water insoluble compounds such as sirolimus and its analogs, including CCI-779, ABT-578 and everolimus, through weeping balloons and catheter injection needles. Sirolimus and its analogs are rapamycins. These liquid formulations increase the apparent solubility of the pharmacologically active but water insoluble compounds by two to four orders of magnitude as compared to the solubility limits of the compounds in water. These liquid formulations rely on the use of a very small amount of organic solvents such as Ethanol and a larger amount of safe amphiphilic (of or relating to a molecule having a polar, water soluble group attached to a non-polar, water insoluble hydration chain) excipients such as polyethylene glycol (PEG 200, PEG 400) and vitamin E TPGS to enhance the solubility of the compounds. These liquid formulations of highly water insoluble compounds are stable and readily flowable at room temperature. Certain excipients, such as Vitamin E TPGS and BHT may be utilized to enhance the storage stability of sirolimus compounds through their anti-oxidation properties.

Table 7, shown below, summarizes the concentrations of the excipient, the co-solvents and the drug for four different liquid formulations. The concentrations of each constituent were determined by liquid chromatography and are presented as weight by volume figures. As may be seen from Table 7, a 4 mg/ml concentration of sirolimus was achieved with an ethanol concentration of two percent, a water concentration of twenty-five percent and a PEG 200 concentration of seventy-five percent.

TABLE 7

| | Formulation B1 | Formulation A1 |
|---|---|---|
| Sirolimus conc. (mg/mL) | 1.79 | 1.0 |
| EtOH conc. (%) | 3.83 | 2 |
| H2O conc. (%) | 7.7 | 25 |
| PEG 200 conc. (%) | 88.5 | 73 |
| Sirolimus conc. (mg/mL) | 2.0 | 4 |
| EtOH conc. (%) | 2.0 | 2.0 |
| H2O conc. (%) | 25 | 25 |
| PEG 200 conc. (%) | 75 | 75 |

As set forth above, a liquid formulation comprising 4 mg/ml of sirolimus may be achieved utilizing PEG 200 as the excipient and ethanol and water as the co-solvents. This concentration of sirolimus is about four hundred to about one thousand times higher than the solubility of sirolimus in water. The inclusion of an effective co-solvent, PEG 200, ensures that the high concentration of sirolimus does not start to precipitate out of solution until diluted five to ten fold with water. The high concentration of sirolimus is necessary to maintain an effective and high local concentration of sirolimus after delivery to the site. The liquid formulations are flowable at room temperature and are compatible with a number of delivery devices. Specifically, each of these formulations were successfully injected through an infusion catheter designated by the brand name CRESCENDO™ from Cordis Corporation, Miami, Fla., as described in more detail subsequently, and the EndoBionics Micro Syringe™ Infusion Catheter available from EndoBionics, Inc., San Leandros, Calif., as described in more detail above, in porcine studies.

Another liquid formulation of sirolimus comprises water and ethanol as co-solvents and Vitamin E TPGS as the excipient. The liquid formulation was created utilizing the following process. Two hundred milligrams of sirolimus and two grams of ethanol were added to a pre-weighed twenty milliliter scintillation vial. The vial was vortexed and sonicated until the sirolimus was completely dissolved. Approximately six hundred milligrams of Vitamin E TPGS was then added to the solution of ethanol and sirolimus. The vial was vortexed again until a clear yellowish solution was obtained. Nitrogen gas was then used to reduce the amount of ethanol in the vial to approximately two hundred twenty-nine milligrams. In a separate vial, three hundred milligrams of Vitamin E TPGS was dissolved in eleven milliliters of purified water while undergoing vortexing. The Vitamin E TPGS and water solution was then added to the first vial containing the sirolimus, Vitamin E TPGS and ethanol. The first vial was then vortexed vigorously and continuously for three minutes. The resulting sirolimus solution was clear with a foam on top. The foam gradually disappeared after sitting at room temperature. An HPLC assay of sirolimus indicated that the sirolimus concentration in the final solution was 15 mg/ml. The final solution had an ethanol concentration of less than two percent, which as stated above is important so as to maintain ethanol as an inactive ingredient. Accordingly, utilizing Vitamin E TPGS as the excipient rather than PEG, resulted in a higher concentration of sirolimus in the final formulation.

Table 8, as shown below, summarizes the composition and visual observations for multiple aqueous formulations of sirolimus utilizing ethanol, Vitamin E TPGS and water at different ratios. The solutions represented by the data contained in Table 8 were generated using essentially the same procedure as described above, except that the ratios between sirolimus and Vitamin E TPGS were varied.

TABLE 8

| | | | | 13.3 ml | |
|---|---|---|---|---|---|
| | | | | water | |
| | | | | containing | |
| Group | Sirolimus | Vitamin E | Ethanol | Vitamin E | Observation of |
| # | mg | TPGS, mg | mg | TPGS, mg | final solution |
| 1 | 202.7 | 642 | 230 | 320 | Clear |
| 2 | 205.2 | 631 | 260 | 330 | Clear |
| 3 | 201.1 | 618 | 260 | 600 | Clear |
| 4 | 204.1 | 625 | 260 | 590 | Clear |
| 5 | 203.3 | 618 | 250 | 1400 | Hazy to clear, |
| | | | | | Viscous |
| 6 | 204.5 | 630 | 250 | 1420 | Clear, viscous |

All of the above preparations except for number five remained as stable solutions at both room temperature and under refrigerated condition. The results in Table 8 indicate that, Vitamin E TPGS may be utilized over a wide range of concentrations to increase the solubility of sirolimus in an aqueous solution.

An aqueous formulation of CCI-779, a sirolimus analog, is prepared utilizing ethanol, Vitamin E TPGS and water. This liquid formulation was made under similar conditions as to that described above. Because of its better solubility in ethanol, only 0.8 grams of ethanol was used to dissolve two hundred milligrams of CCI-779 as opposed to the two grams of sirolimus. After the amount of ethanol was reduced to approximately two hundred thirty milligrams, eleven milliliters of purified water containing three hundred milligrams of Vitamin E TPGS was added to the vial of ethanol and CCI-779. The combined solution was vortexed for three minutes and resulted in a clear solution. An HPLC assay of CCI-779 indicated that the concentration of CCI-779 in the final solution was 15 mg/ml. The concentration of ethanol in the final solution was less than two percent. Accordingly, the results are substantially identical to that achieved for the sirolimus.

As stated above, a number of catheter-based delivery systems may be utilized to deliver the above-described liquid formulations. One such catheter-based system is the CRESCENDO™ infusion catheter. The CRESCENDO™ infusion catheter is indicated for the delivery of solutions, such as heparinized saline and thrombolytic agents selectively to the coronary vasculature. The infusion catheter may also be utilized for the delivery of the liquid formulations, including the liquid solution of sirolimus, described herein. The infusion region includes an area comprised of two inflatable balloons with multiple holes at the catheter's distal tip. The infusion region is continuous with a lumen that extends through the catheter and terminates at a Luer port in the proximal hub. Infusion of solutions is accomplished by hand injection through an infusion port. The catheter also comprises a guidewire lumen and a radiopaque marker band positioned at the center of the infusion region to mark its relative position under fluoroscopy.

A larger amount of safe amphiphilic excipients, such as Vitamin E TPGS, PEG 200, and PEG 400, may be used alone or in combination to enhance the solubility and stability of the drug during the preparation of the formulations. Vitamin E TPGS may also enhance the drug transfer into the local tissues during the deployment of the medical device and contact with a vascular tissue. Enhanced transfer of the drug from the external surfaces and subsequent deposition of the drug in the local tissue provide for a long-term drug effects and positive efficacy such as reduced neointimal formation after an angioplasty procedure or a stent implantation. In addition to improving the solubility of a water-insoluble drug during the formulation preparation, these excipients may also help form a non-crystalline drug formulation on a device surface when the water is substantially dried off, and facilitate a fast detachment of the drug formulation from the coating of a medical device when contacted with a local tissue.

Separately, a series of aqueous injectable formulations were developed for the local or regional delivery of taxanes for the treatment of coronary artery disease. Taxanes include paclitaxel and docetaxel. In one preferred embodiment of the invention, the therapeutic agent is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216,-1993). “Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol(2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-.gamma.-aminobutyryltaxol formate, 2′-acetyl taxol, 7-acetyl taxol, 7-glycine carbamate taxol, 2′-OH-7-PEG(5000)carbamate taxol, 2′-benzoyl and 2′,7-dibenzoyl taxol derivatives, other prodrugs (2′-acetyl taxol; 2′,7-diacetyltaxol; 2′ succinyltaxol; 2′-(beta-alanyl)-taxol); 2′ gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).

As described above, it is generally very difficult to create aqueous solution formulations of water insoluble and lipophilic drugs such as paclitaxel, including analogs and derivatives, without resorting to substantial amounts of surfactants, co-solvents and the like. Typically, excipients such as Tween 20, Tween 80, cremaphor and polyethylene glycol have varying degrees of toxicity relative to the surrounding tissue. Accordingly, the use of these agents and organic co-solvents such as DMSO, NMP and ethanol need to be minimized to reduce the toxicity of the solution relative to the surrounding tissue. Essentially, the key to a successful injectable formulation of a water insoluble compound is to find a good combination or balance of excipient and co-solvent and an optimal range of the additives in the final dosage form to balance the improvement of drug solubility and necessary safety margin.

A series of aqueous injectable formulations of paclitaxel are disclosed herein for local or regional delivery through weeping balloons, catheter injection needles and other catheter-based delivery systems as described herein. Such injectable formulations make it possible for the delivery of pharmaceutically active but water insoluble compounds through a catheter-based device. The injectable formulations may be aqueous solutions or suspensions depending on the dosage. In these formulations, the solubility of the drug may be increased by several orders of magnitude compared to the solubility limits of the compounds in water.

These injectable formulations rely on the use of a very small amount of organic solvents, such as ethanol (typically less than two percent), and a larger amount of safe amphiphilic excipients, such as PEG 200, PEG 400 and Vitamin E TPGS, to enhance the solubility of the drug. These injectable formulations of highly water insoluble compounds are stable and readily flowable at room temperature. Some excipients, including Vitamin E, Vitamin E TPGS and BHT may also be utilized to enhance the storage stability of the paclitaxel or other taxane compounds through their anti-oxidation properties as more fully described herein. Alternately, stable suspensions or emulsions of water insoluble compounds may be formed utilizing similar solubility-enhancing agents to obtain a higher drug concentration for local or regional injections. The pH value of these suspensions or emulsions may be adjusted to improve the stability of the formulations. These suspension formulations may be more likely to maintain a more sustained release for the drug at the injection site as compared with the solution formulations.

Table 9, shown below, summarizes a number of injectable liquid formulations of paclitaxel utilizing combinations of ethanol, PEG 400 and water. Specifically, the formulations set forth in Table 9 were made and analyzed for their concentrations of its various constituents. The concentrations are determined by liquid chromatography and are presented as weight by volume figures. The concentration of ethanol is preferably two or less percent so as to avoid ethanol becoming an active ingredient in the formulation. With the concentration of paclitaxel at 0.5 mg/ml and a PEG 400 concentration of fifty percent, the final solution has a medium viscosity. Higher concentrations of PEG 400 and paclitaxel resulted in more viscous solutions. When the concentration of paclitaxel is greater than 1 mg/ml and the solution is diluted with pure water, the paclitaxel precipitates out of solution. Each of these formulations may be successfully injected through the Cordis CRESCENDO™ infusion catheter and the EndoBionics Micro Syringe™ infusion catheter.

TABLE 9

| | Paclitaxel conc. | Ethanol conc. | PEG 400 | Observation of |
|---|---|---|---|---|
| Group # | (mg/ml) | (mg/ml) | (%) | final solution |
| 1 | 0.5 | 0 | 50 | Medium viscosity |
| 2 | 0.5 | 0 | 100 | Viscous |
| 3 | 1 | 0 | 100 | Viscous |
| 4 | 5 | 2 | 100 | Viscous |

Another aqueous liquid or injectable formulation of paclitaxel is made utilizing ethanol, PEG 400 and water, and ethanol, Vitamin E TPGS, PEG400 and water. In making the first formulation, 100 mg of paclitaxel is added to 400 μl of ethanol in a pre-weighed 20 ml scintillation vial. The mixture of paclitaxel and ethanol is vortexed and heated in a 60 degree C. bath for ten minutes. Once the drug is completely solubilized, 20 ml of PEG 400 is then added to make the final paclitaxel concentration 5 mg/ml. This solution remained clear. In a separate experiment, a series of 20 ml scintillation vials containing Vitamin E TPGS are heated or warmed up in a 50 degree C. water bath for ten minutes. Concurrently, distilled water is also warmed in a 50 degree C. water bath. Once the Vitamin E TPGS was melted in each vial, the distilled water is added into the Vitamin E TPGS vials and vortexed for one minute and left to stand in the water bath for two hours. The final concentrations of Vitamin E TPGS in water were one, five and fifteen percent. The paclitaxel stock solution (5 mg/ml) described herein was then mixed with the Vitamin E TPGS solutions to make the final paclitaxel formulations. The results are listed in Table 10 given below. In a preferred embodiment, the solution comprises 1.25 mg/ml paclitaxel, 3.75 percent Vitamin E TPGS, 0.5 percent ethanol and twenty-five percent PEG 400. This solution is clear and has a low viscosity and thus may be easily utilized with catheter-based systems.

TABLE 10

| | Paclitaxel | Vitamin E | Ethanol | PEG | |
|---|---|---|---|---|---|
| | conc. | TPGS conc. | conc. | 400 | Observation of |
| Group # | (mg/ml) | (%) | (%) | (%) | final solution |
| 1 | 1.25 | 3.75 | 0.5 | 25 | Clear, low viscosity |
| 2 | 1.7 | 5.0 | 0.7 | 33 | Clear, med viscosity |
| 3 | 2.5 | 7.5 | 1.0 | 50 | Clear, med viscosity |
| 4 | 5 | 0 | 2 | 100 | Clear, viscous |

Other aqueous formulations of paclitaxel utilizing ethanol, Vitamin E TPGS and water were made at different ratios. The formulations were made utilizing the same procedure as described above with the exception that PEG 400 was omitted from the formulations. The compositions and observations for the final solution are set forth in Table 11 given below. All of the preparations set forth in Table 11 were clear solutions upon mixing and vortexing. Once the temperature of the solution gradually cooled down to room temperature, all formulations except that from group number one became a cloudy suspension of paclitaxel and Vitamin E TPGS.

TABLE 11

| | Paclitaxel | Vitamin E | Ethanol | |
|---|---|---|---|---|
| | conc. | TPGS | conc. | Observation of |
| Group # | (mg/ml) | conc. (%) | (%) | final formulation |
| 1 | 1 | 7.5 | 2 | Hazy to Clear |
| 2 | 5 | 7.5 | 2 | Stable suspension |
| 3 | 10 | 7.5 | 2 | Stable suspension |
| 4 | 15 | 7.5 | 2 | Stable suspension |

The utility of such an injectable paclitaxel suspension is that it may be injected through an EndoBionics Micro Syringe™ infusion catheter and potentially provide a more sustained release of paclitaxel from the injection site. With the presence of precipitated Vitamin E TPGS, the toxicity of paclitaxel will likely be lessened as well. Other excipients such as additional anti-oxidants and stabilizers may also be added to the formulation to increase the shelf life without significantly altering the properties of the formulations.

As may be seen from the above data, true aqueous liquid formulations of paclitaxel were made for up to 2.5 mg/ml, which is about 1000 fold higher than the solubility of paclitaxel in water. The inclusion of an effective co-solvent, PEG 200/PEG 400, functions to prevent such a high concentration of paclitaxel from precipitating out of solution until diluted five to ten fold. Such a high concentration is preferred so as to maintain an effective and high local concentration of paclitaxel after delivery to the local site with a small injection volume. The solution formulation is flowable at room temperature, and as set forth herein, is compatible with any number of catheter-based delivery systems. The viscosity of the injectable formulation can be adjusted by changing the mixture ratio of PEG and Vitamin E TPGS. Also, additional excipients may be included without substantially affecting the viscosity of the final injection solution. Viscosity is the key to minimizing the potential damage of the arterial wall at the site of the injection.

It is important to note that the concept of injectable formulations may be oriented to other taxane compounds. For example, any paclitaxel analogs may be formulated using the disclosed agents and methodologies. Depending on the water solubility of the compound, a wide range of safe solvent and excipient selections and amounts such as acetone, cyclodextrin can be selected to optimize the formulation. Anti-oxidative compounds such as Vitamin E mixtures, Vitamin E TPGS and BHT can be used to increase the storage stability of the liquid formulations. Amounts of formulations excipients such as mannitol, sucrose, trehelose, may be used to produce stable lyophilized formulations. Amounts of amphiphilic compounds such as Vitamin E TPGS can be adjusted to modulate the tissue diffusion and retention of the drug after local delivery.

In addition to infusion catheters, these liquid formulations of highly water insoluble compounds are stable and may be used for coating an external surface of a medical device such as a PTCA balloon.

Alternately, stable solutions, suspensions or emulsions of water insoluble compounds may be formed utilizing similar solubility-enhancing agents to obtain a higher drug concentration than the formulations set forth above for coating the external surfaces of a medical device. The pH value of these suspensions or emulsions may be adjusted to improve the stability of the drug formulations.

The viscosity of the liquid formulations can be adjusted by changing the mixture ratio of PEG and Vitamin E TPGS. Also, additional excipients may be included without substantially affecting the viscosity of the final coating solution but improve the stability of the drug in the formulation and coating.

Although anti-restenotic agents have been primarily described herein, the present invention may also be used to deliver other agents alone or in combination with anti-restenotic agents. Some of the therapeutic agents for use with the present invention which may be transmitted primarily luminally, primarily murally, or both and may be delivered alone or in combination include, but are not limited to, antiproliferatives, antithrombins, immunosuppressants including sirolimus, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, bivalirudin, phenoxodiol, etoposide, ticlopidine, dipyridamole, and trapidil alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting, and vasodilation.

Anti-inflammatories include but are not limited to non-steroidal anti-inflammatories (NSAID), such as aryl acetic acid derivatives, e.g., Diclofenac; aryl propionic acid derivatives, e.g., Naproxen; and salicylic acid derivatives, e.g., Diflunisal. Anti-inflammatories also include glucocoriticoids (steroids) such as dexamethasone, aspirin, prednisolone, and triamcinolone, pirfenidone, meclofenamic acid, tranilast, and nonsteroidal anti-inflammatories. Anti-inflammatories may be used in combination with antiproliferatives to mitigate the reaction of the tissue to the antiproliferative.

The agents may also include anti-lymphocytes; anti-macrophage substances; immunomodulatory agents; cyclooxygenase inhibitors; anti-oxidants; cholesterol-lowering drugs; statins and angiotens in converting enzyme (ACE); fibrinolytics; inhibitors of the intrinsic coagulation cascade; antihyperlipoproteinemics; and anti-platelet agents; anti-metabolites, such as 2-chlorodeoxy adenosine (2-CdA or cladribine); immuno-suppressants including sirolimus, everolimus, tacrolimus, etoposide, and mitoxantrone; anti-leukocytes such as 2-CdA, IL-1 inhibitors, anti-CD116/CD18 monoclonal antibodies, monoclonal antibodies to VCAM or ICAM, zinc protoporphyrin; anti-macrophage substances such as drugs that elevate NO; cell sensitizers to insulin including glitazones; high density lipoproteins (HDL) and derivatives; and synthetic facsimile of HDL, such as lipator, lovestatin, pranastatin, atorvastatin, simvastatin, and statin derivatives; vasodilators, such as adenosine, and dipyridamole; nitric oxide donors; prostaglandins and their derivatives; anti-TNF compounds; hypertension drugs including Beta blockers, ACE inhibitors, and calcium channel blockers; vasoactive substances including vasoactive intestinal polypeptides (VIP); insulin; cell sensitizers to insulin including glitazones, P par agonists, and metformin; protein kinases; antisense oligonucleotides including resten-NG; antiplatelet agents including tirofiban, eptifibatide, and abciximab; cardio protectants including, VIP, pituitary adenylate cyclase-activating peptide (PACAP), apoA-I milano, amlodipine, nicorandil, cilostaxone, and thienopyridine; cyclooxygenase inhibitors including COX-1 and COX-2 inhibitors; and petidose inhibitors which increase glycolitic metabolism including omnipatrilat. Other drugs which may be used to treat inflammation include lipid lowering agents, estrogen and progestin, endothelin receptor agonists and interleukin-6 antagonists, and Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic surfactants may be used. Examples of nonionic excipients include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEO) and polyvinyl pyrrolidone (PVP); carboxylic acids including D-lactic acid, glycolic acid, and propionic acid; surfactants with affinity for hydrophobic interfaces including n-dodecyl-.beta.-D-maltoside, n-octyl-.beta.-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; (PC) CM-PEG; cholic acid; sodium dodecyl sulfate (SDS); docusate (AOT); and taumocholic acid.

Although antioxidants may be utilized with any number of drugs, including all the drugs described herein, exemplary embodiments of the invention are described with respect to rapamycin and more specifically, drug eluting implantable medical devices comprising rapamycin. As briefly set forth above, molecules or specific portions of molecules may be particularly sensitive to oxidation. In rapamycins, the conjugated triene moiety of the molecule is particularly susceptible to oxidation. Essentially, oxygen breaks the carbon chain of the conjugate triene moiety and the bioactivity of the rapamycin is degraded. In addition, as is typical with oxidation processes, the drug is broken down into one or more different compounds. Accordingly, it may be particularly advantageous to mix or co-mingle an antioxidant with the rapamycin. Specifically, in order to achieve the best results, it is important to co-mingle the antioxidant and the drug to the greatest extent possible. More importantly, the physical positioning of the antioxidant proximate to the drug is the key to success. The antioxidant preferably remains free to combine with oxygen so that the oxygen does not break up the moiety and ultimately degrade the drug. Given that the rapamycin may be incorporated into a polymeric coating or matrix, it is particularly important that the antioxidant be maintained proximate to the drug rather than the polymer(s). Factors that influence this include the constituents of the polymeric matrix, the drug, and how the polymer/drug coating is applied to the implantable medical device. Accordingly in order to achieve the desired result, selection of the appropriate antioxidant, the process of mixing all of the elements and the application of the mixture is preferably tailored to the particular application.

A number of antioxidants were tested to determine their efficacy in preventing the degradation of rapamycin, or more specifically, sirolimus. Screening experiments were performed to evaluate the solubility of various antioxidants in tetrahydroxyfuran (THF) solutions containing sirolimus and the percentage of antioxidant required to prevent oxidation of sirolimus alone and in a basecoat polymeric matrix. THF is the solvent in which sirolimus may be dissolved. It is important to note that other solvents may be utilized. Two sets of controls were utilized. Control #1 comprises solutions of THF and sirolimus and/or polymers with no antioxidant, and Control #2 comprises solutions of THF and sirolimus and/or polymers, wherein the THF contains a label claim of 250 ppm of BHT as a stabilizer from the vendor of THF. In other words, the BHT is an added constituent of the THF solvent to prevent oxidation of the solvent. Table 12 shown below is a matrix of the various mixtures. All percentages are given as weight/volume.

TABLE 12

| | | Antioxidant | Target | Antioxidant |
|---|---|---|---|---|
| | Target | Grams/ | % | Grams/ |
| Antioxidant | % Antioxidant | 50 mL | Antioxidant | 50 mL |
| Ascorbic | 0.02 | 0.01 | 0.5 | 0.25 |
| Acid | | | | |
| Ascorbyl | 0.01 | 0.005 | 0.02 | 0.01 |
| Palmitate | | | | |
| BHT | 0.005 | 0.0025 | 0.02 | 0.01 |
| Tocopherol | 0.05 | 0.025 | 0.075 | 0.0375 |
| Control #1 | 0.0 | 0.0 | 0.0 | 0.0 |
| Control #2 | 250 ppm BHT | 0.0 | 0.0 | 0.0 |

Table 13, shown below, identifies the samples for evaluation. All percentages are given as weight/volume. The samples in Table 13 contain no polymer. Table 14, also shown below, identifies the samples for evaluation with the solutions now comprising polymers, including PBMA and PEVA.

TABLE 13
Solutions with Sirolimus Only-No Polymers

| SAMPLE ID # | ACTUAL % ANTIOXIDANT |
|---|---|
| AA1A | 0.026 Ascorbic Acid |
| AA2A | 0.50 Ascorbic Acid |
| AP1A | 0.01 Ascorbyl Palmitate |
| AP2A | 0.02 Ascorbyl Palmitate |
| BHT1A | 0.006 BHT |
| BHT2A | 0.02 BHT |
| C2A | Control #2 - 250 ppm BHT |
| TP1A | 0.048 Tocopherol |
| TP2A | 0.082 Tocopherol |
| C1A | Control #1 |

TABLE 14
Solutions with Sirolimus and Polymers

| SAMPLE ID # | ACTUAL % ANTIOXIDANT |
|---|---|
| AA1B | 0.022 Ascorbic Acid |
| AA2B | 0.508 Ascorbic Acid |
| AP1B | 0.01 Ascorbyl Palmitate |
| AP2B | 0.02 Ascorbyl Palmitate |
| BHT1B | 0.006 BHT |
| BHT2B | 0.02 BHT |
| C2B | Control #2 - 250 ppm BHT |
| TP1B | 0.054 Tocopherol |
| TP2B | 0.102 Tocopherol |
| C1B | Control #1 |

As set forth above, each of the samples in Tables 13 and 14 were tested to determine the solubility of the various antioxidants as well as their effectiveness in preventing drug degradation. All of the antioxidants were soluble in both the solvent with sirolimus solutions and the solvent with sirolimus and polymer solutions. The solubility of each of the antioxidants was determined by a visual inspection of the test samples.

Table 15, as shown below, identifies the chosen samples that were evaluated for drug content (percent label claim or % LC) after five (5) days in an oven set at a temperature of sixty degrees C. (60° C.). The samples were evaluated after five (5) days utilizing a drug testing assay for sirolimus. In the exemplary embodiment, a HPLC assay was utilized. The important numbers are the percent label claim number (% LC) of the solutions that indicates how much of the drug remains or is recovered. The antioxidants, BHT, Tocopherol, and/or Ascorbic Acid provided significant protection against the harsh environmental conditions of the test. Lower % LC numbers are evident in solutions samples that do not contain an antioxidant.

TABLE 15
Solutions with Sirolimus and Polymers after 5 days 60° C. storage

| SAMPLE ID # | ACTUAL % ANTIOXIDANT | % LC |
|---|---|---|
| AA2B | 0.508 Ascorbic Acid | 96.4 |
| AP2B | 0.02 Ascorbyl Palmitate | 82.5 |
| BHT2B | 0.02 BHT | 94.8 |
| TP2B | 0.102 Tocopherol | 97.3 |
| C2B | Control #2 - 250 ppm BHT | 99.5 |
| C1B | Control #1 | 70.0 |
| C1B | Control #1 | 69.2 |

As shown below, Table 16 provides the % LC results for the samples without polymers and Table 17 provides the % LC results for the samples with polymer after four (4) weeks of sixty degrees C. (60° C.).

TABLE 16

| | CALCULATED | THEORETICAL | |
|---|---|---|---|
| SAMPLE | RESULTS | CONCENTRATION | |
| ID # | (μg/ml) | (μg/ml) | % LC |
| AA1A | 1155.56 | 1669.2 | 69.2 |
| AA2A | 1280.90 | 1669.2 | 76.7 |
| AP1A | 851.45 | 1669.2 | 51.0 |
| AP2A | 939.36 | 1669.2 | 56.3 |
| BHT1A | 437.38 | 1669.2 | 26.2 |
| BHT2A | 1434.98 | 1669.2 | 86.0 |
| TP1A | 1335.58 | 1669.2 | 80.0 |
| TP2A | 1618.61 | 1669.2 | 97.0 |
| C1A #1 | 608.64 | 1669.2 | 36.5 |
| C1A #2 | 552.57 | 1669.2 | 33.1 |
| C2A #1 | 1794.70 | 1669.2 | 107.5 |
| C2A #2 | 1794.67 | 1669.2 | 107.5 |

TABLE 17

| | CALCULATED | THEORETICAL. | |
|---|---|---|---|
| SAMPLE | RESULTS | CONCENTRATION | |
| ID # | (μg/ml) | (μg/ml) | % LC |
| AA1B | 884.95 | 1669.2 | 53.0 |
| AA2B | 1489.70 | 1669.2 | 89.2 |
| AP1B | 743.98 | 1669.2 | 44.6 |
| AP2B | 906.76 | 1669.2 | 54.3 |
| BHT1B | 595.18 | 1669.2 | 35.7 |
| BHT2B | 1396.55 | 1669.2 | 83.7 |
| TP1B | 1177.30 | 1669.2 | 70.5 |
| TP2B | 1695.45 | 1669.2 | 101.6 |
| C1B #1 | 490.56 | 1669.2 | 29.4 |
| C1B #2 | 470.15 | 1669.2 | 28.2 |
| C2B #1 | 1807.44 | 1669.2 | 108.3 |
| C2B #2 | 1810.41 | 1669.2 | 108.5 |

As seen from a review of the % LC or drug recovery enumerated in Tables 16 and 17, higher percent concentrations of Tocopherol, BHT, and/or Ascorbic Acid provide significant protection against the harsh environmental conditions of the test. However, higher % LC numbers are evident in all controls containing 250 ppm BHT due to possible solution evaporation of the samples from loose caps on the samples in the 60° C. storage condition.

Additional samples were tested under ambient conditions, rather than at 60° C., and using the same compositions; however, the test period was expanded to seven weeks. The results are given in Table 18, shown below.

TABLE 18

| | CALCULATED | THEORETICAL | |
|---|---|---|---|
| SAMPLE | RESULTS | CONCENTRATION | |
| ID # | (μg/ml) | (μg/ml) | % LC |
| C1A | 1248.04 | 1669.2 | 74.8 |
| C2A | 1578.15 | 1669.2 | 94.5 |
| C1BMS | 1376.46 | 1669.2 | 82.5 |
| C1BMS | 1377.20 | 1669.2 | 82.5 |
| C2B | 1633.07 | 1669.2 | 97.8 |
| TP1A | 1635.54 | 1669.2 | 98.0 |
| TP2A | 1632.05 | 1669.2 | 97.8 |
| TP1B | 1631.75 | 1669.2 | 97.8 |
| TP2B | 1621.64 | 1669.2 | 97.2 |
| AA1A | 1590.17 | 1669.2 | 95.3 |
| AA2A | 1578.21 | 1669.2 | 94.5 |
| AA1B | 1598.79 | 1669.2 | 95.8 |
| AA2B | 1592.47 | 1669.2 | 95.4 |
| AP1A | 1429.76 | 1669.2 | 87.7 |
| AP2A | 1415.83 | 1669.2 | 84.8 |
| AP1B | 1472.45 | 1669.2 | 88.2 |
| AP2B | 1480.31 | 1669.2 | 88.7 |
| BHT1A | 1527.18 | 1669.2 | 91.5 |
| BHT2A | 1601.72 | 1669.2 | 96.0 |
| BHT1B | 1579.50 | 1669.2 | 94.6 |
| BHT2B | 1614.52 | 1669.2 | 96.7 |

As may be seen from a review of Table 18, the results are substantially similar to those obtained for five (5) days and four (4) weeks at sixty degrees C. (60° C.) % LC data. Accordingly, in a preferred exemplary embodiment, Tocopherol, BHT and/or Ascorbic Acid may be utilized to substantially reduce drug degradation due to oxidation.

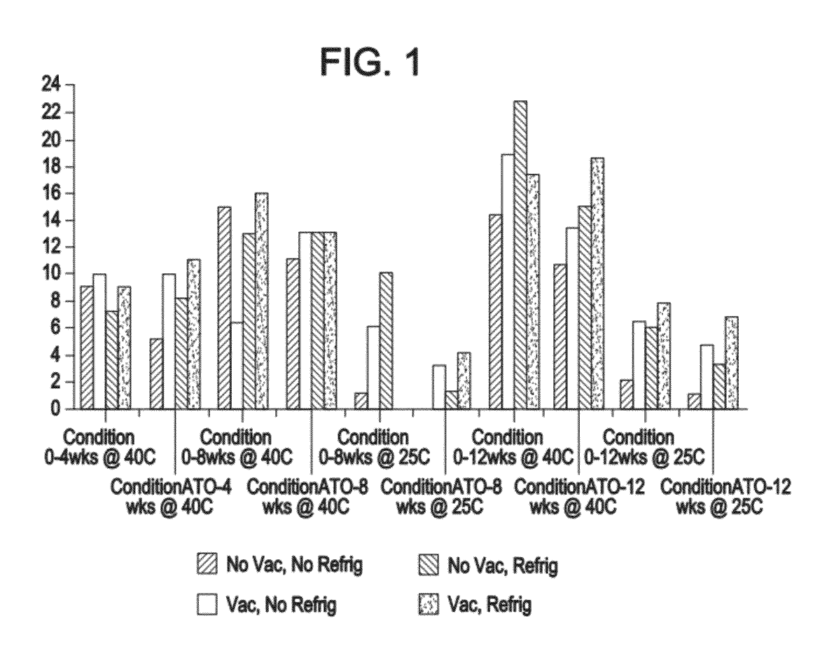

Referring to FIG. 1, there is illustrated in graphical format, the results of the same drug screening as described above with the solution applied to a cobalt-chromium, 18 mm stent. In this test, two sets of solution samples were utilized, one with sirolimus and polymer solution containing the antioxidant and one with sirolimus and polymer solution containing no antioxidant. The antioxidant utilized was 0.02 weight percent BHT per total basecoat solids. The test was utilized to determine the percent drug content change over a time period of 0 to 12 weeks under two conditions; namely, 40° C. with 75 percent relative humidity, and ambient conditions (25° C.). As can be seen from the chart, the addition of BHT to the solution lessens drug degradation at both 8 weeks and 12 weeks under ambient conditions. Accordingly, if one does not stabilize the base coat solution, other process techniques must be utilized; namely, refrigeration and/or vacuum drying.

In accordance with another exemplary embodiment, balloons or other inflatable or expandable devices may be temporarily positioned within a body to deliver a therapeutic agent and/or combination of therapeutic agents and then removed. The therapeutic agents may include liquid formulations of rapamycins as described above or any other formulations thereof. This type of delivery device may be particularly advantageous in the vasculature where stents may not be suitable, for example, in the larger vessels of the peripheral vascular system and at bifurcation points in the vasculature, or where the long term scaffolding of a stent is not required or desired.

In use, the balloon or other inflatable or expandable device may be coated with one or more liquid formulations of therapeutic agents(s) and delivered to a treatment site. The act of inflation or expansion would force the therapeutic agents into the surrounding tissue. The device may be kept in position for a period of between ten seconds to about five minutes depending upon the location. If utilized in the heart, shorter durations are required relative to other areas such as the leg.

The balloon or other inflatable device may be coated in any suitable manner including dipping and spraying as described above. In addition, various drying steps may also be utilized. If multiple coats are required for a specific dosage, then additional drying steps may be utilized between coats.

In addition to the solubility enhancers and the organic solvents described herein, other antioxidant excipients may also be used in the formulations to stabilize the pharmaceutical agents, such as sirolimus (rapamycin), in the coating. Such antioxidants include BHT, BHA, vitamin E, vitamin E TPGS, ascorbic acid (vitamin C), ascorbyl palmitate, ascorbyl myristate, resveratrol and its many synthetic and semi-synthetic derivatives and analogs, etc. These antioxidant excipients may also serve additional functions such as facilitating the release of drug coatings from the balloon surface upon contact with the artery wall. These and other similar excipients will remain in the coating after the drying processes and serve to speed up the drug in the coating from detaching from the balloon surface at the disease site. The enhancement of drug coating separation from the balloon through the use of these agents is possibly caused by their inherent tendency to absorb water upon placement in the physiological situation such as inside the arteries. The swelling and physical expansion of the coating at the delivery site will help increase the delivery efficiency of the drug coating into the diseased arterial tissue. Depending on the nature of the particular excipients they may also have the added benefits of enhancing the drug transport from the coating into the diseased cells and the tissues. For instance, vasodilators such as cilostazol and dipyridamole, may also be used as excipients to improve the intracellular transport of the drugs. Also certain excipients may also enhance the cross-membrane transport and even sequestration of the drugs into the local tissues.

The balloon coating conditions may also play important roles in creating the optimal morphology of the final drug coating in that the drying speed of the drug coating matrix on the balloons, the exposure time of subsequent coating time (second, third, fourth coatings, etc. if needed) may re-dissolve the previously laid coating layers. A variation of the current invention is that coating formulations with gradually increasing water content may be used in subsequent coating steps to minimize the coatings laid down previously and increase coating weight and uniformity of each coating step. The final coating solution may even be an emulsion (high water content, and/or high drug content) as opposed to clear aqueous solutions (high organic solvent content) to complete the coating processes.

The following experiments were included to illustrate the principles and formulations used in creating the disclosed aqueous liquid formulations of sirolimus and paclitaxel for local delivery. Many of the excipients may be interchanged to enhance one aspect or another of the formulations, without affecting the efficacy of the particular formulation.

In a first experiment, an aqueous coating solution using PEG 400 and BHT as the solubility and transport enhancers was formulated. To a tared 10-ml scintillation vial was added about 100.5 mg of sirolimus (rapamycin, stock #124623500 batch #RB5070)), followed by about 9.8 mg of PEG 400 (Aldrich), and 10.1 mg of BHT (Aldrich). One ml of ethanol was then added to dissolve the above components under shaking. Once the solution became completely clear, 1-ml of water was slowly added to the solution. The mixed solution became cloudy and sirolimus in the organic solution was immediately precipitated out. Sirolimus remained insoluble upon agitation. The composition of the coating formulation is shown in Table 19.

TABLE 19
Aqueous coating solution using PEG 400, BHT (A1 formulation)

| | | Actual amt |
|---|---|---|
| | Formulation | in 2 mL |
| | A1 | solution |
| Sirolimus conc | 50 | 100.5 mg |
| (mg/ml) | | |
| PEG 400 (mg/ml) | 5 | 9.8 mg |
| BHT (mg/ml) | 5 | 10.1 mg |
| EtOH (%) | 50 | 1 ml |
| H2O (%) | 50 | 1 ml |

No further experimentation on this particular formula was done because of the insolubility of the sirolimus.

In a second experiment, an aqueous coating solution using PEG 400 and BHT as the solubility and transport enhancers was formulated. To a tared 10-ml scintillation vial was added about 99.0 mg of sirolimus (rapamycin, stock #124623500 batch #RB5070)), followed by about 10.1 mg of PEG 400 (Aldrich), and 9.9 mg of BHT (Aldrich). One and half ml (1.5 ml) of ethanol was then added to dissolve the above components under shaking. Once the solution became completely clear, 0.5-ml of water was slowly added to the solution. The mixed solution remained clear and stable upon agitation. The composition of the coating formulation is shown in Table 20.

TABLE 20
Aqueous coating solution using PEG 400, BHT (A3)

| | | Actual amt | |
|---|---|---|---|
| | Formulation | in 2 mL | |
| | A3 | solution | |
| Sirolimus conc | 50 | 99 | mg |
| (mg/ml) | | | |
| PEG 400 (mg/ml) | 5 | 10.1 | mg |
| BHT (mg/ml) | 5 | 9.9 | mg |
| EtOH (%) | 75 | 1.5 | ml |
| H2O (%) | 25 | 0.5 | ml |

The clear solution formulation of Table 20 was transferred to a glass slide for coating morphology studies. A Gilson pipetteman was used to transfer 20 ul of the coating solution onto a pre-weighed glass slide three times. The coating spots on the slides were allowed to dry at room temperature in a laminar hood. The coating spots gradually become opaque after drying. The weight of the slides with coated spots were measured and recorded in lines 1 and 4 of Table 21. The drug content transfer efficiency of the coating solution was determined to be approximately 95 percent.

TABLE 21
Coating formulations and weight of coated glass slides

| | Tare | | coating | | coating | | | | |
|---|---|---|---|---|---|---|---|---|---|
| Glass | weight | wt after | weight | coat wt | solution | | theor | Transfer | |
| slide # | (g) | coating | (g) | in mg | vol (ul) | | amt (mg) | eff (%) | Note |
| 1 (A3) | 4.7626 | 4.7653 | 0.0027 | 2.70 | 3 × 20 | ul | 2.85 | 94.7 | clear |
| | | | | | | | | | solution |
| 2 (B1) | 4.7614 | 4.7640 | 0.0026 | 2.60 | 3 × 20 | ul | 2.85 | 91.2 | stable |
| | | | | | | | | | emulsion |
| 3 (B1) | 4.7444 | 4.7491 | 0.0047 | 4.70 | 100 | ul | 4.75 | 98.9 | stable |
| | | | | | | | | | emulsion |
| 4 (A3) | 4.7665 | 4.7714 | 0.0049 | 4.90 | 100 | ul | 4.95 | 99.0 | clear |
| | | | | | | | | | solution |
| 5 (A5) | 4.7666 | 4.7689 | 0.0023 | 2.30 | 3 × 20 | ul | 3.03 | 75.9 | partial |
| | | | | | | | | | precipitation |
| 6 (C1) | 4.7347 | 4.7371 | 0.0024 | 2.40 | 50 | ul | 2.51 | 95.6 | clear |
| | | | | | | | | | solution |
| 7 (A5) | 4.7367 | 4.7397 | 0.003 | 3.00 | 100 | ul | 5.05 | 59.4 | partial |
| | | | | | | | | | precipitation |
| 8 | 4.8726 | discarded | | | | | | | |
| 9 | 4.7716 | 4.7739 | 0.0023 | 2.30 | 50 | ul | 2.38 | 96.6 | stable |
| (B1) | | | | | | | | | emulsion |
| 10 | 4.7646 | 4.7742 | 0.0096 | 4.80 | 100 | ul | 5.05 | 95.0 | clear |
| (C1) | | | | | | | | | solution |

In a third experiment, an aqueous coating solution using PEG 400 and BHT as the solubility and transport enhancers was formulated. To a tared 10-ml scintillation vial was added about 101.0 mg of sirolimus (rapamycin, stock #124623500 batch #RB5070)), followed by about 10.0 mg of PEG 1000 (Aldrich), and 10.2 mg of BHT (Aldrich). One point three ml (1.3 ml) of acetone was then added to dissolve the above components under shaking. Once the solution became completely clear, 0.7-ml of water was slowly added to the solution. The mixed solution immediately became cloudy. Upon agitation, part of the drug precipitated out of the solution and stuck to the vial wall. The composition of the coating formulation is shown in Table 22.

TABLE 22
Aqueous coating formulation using PEG 1000, BHT (A5)

| | | Actual am |
|---|---|---|
| | Formulation | in 2 mL |
| | A5 | solution |
| Sirolimus conc | 50 | 101.0 |
| (mg/ml) | | |
| PEG 1000 (mg/ml) | 5 | 10.0 |
| BHT (mg/ml) | 5 | 10.2 |
| EtOH (%) | 65 | 1.3 |
| H2O | 35 | 0.7 |

The clear portion of the solution of the formulation of Table 22 was transferred to a glass slide for coating morphology studies. A Gilson pipetteman was used to transfer 20 ul of the coating solution onto a pre-weighed glass slide three times. The coating spots on the slides were allowed to dry at room temperature in a laminar hood. The coating spots gradually become opaque after drying. The weight of the slides with coated spots were measured and recorded in lines 5 and 7 of Table 18. The drug content transfer efficiency of the coating solution was determined to be approximately 76 percent. The decreased efficiency of drug transfer was mostly like caused by the precipitation of sirolimus from the solution upon the addition of water. This formulation is not suitable for coating since the weight of final coating is not easily controlled.

In a fourth experiment, an aqueous coating solution using PEG 400 and BHT as the solubility and transport enhancers was formulated. To a tared 10-ml scintillation vial was added about 95.5 mg of sirolimus (rapamycin, stock #124623500 batch #RB5070)), followed by about 9.9 mg of PEG 400 (Aldrich), and 10.2 mg of BHT (Aldrich). One point two ml (1.2 ml) of acetone was then added to dissolve the above components under shaking. Once the solution became completely clear, 0.8-ml of water was slowly added to the solution. The mixed solution immediately became cloudy and remained as a stable emulsion at room temperature. The composition of the coating formulation is shown in Table 23.

TABLE 23
Aqueous coating formulation using PEG 400, BHT (B1)

| | | actual am in |
|---|---|---|
| | Formulation | 2 mL |
| | B1 | solution |
| Sirolimus conc | 50 | 95.5 |
| (mg/ml) | | |
| PEG 400 (mg/ml) | 5 | 9.9 |
| BHT (mg/ml) | 5 | 10.2 |
| Acetone (%) | 60 | 1.2 |
| H2O (%) | 40 | 0.8 |

The stable emulsion of the formulation of Table 23 was transferred to a glass slide for coating morphology studies. A Gilson pipetteman was used to transfer 20 ul of the coating solution onto a pre-weighed glass slide three times. The coating spots on the slides were allowed to dry at room temperature in a laminar hood. The coating spots gradually become opaque after drying. The weight of the slides with coated spots were measured and recorded in line 2 of Table 21. Coating solution B1 was similarly transferred to glass slides with various amounts, with the results recorded in lines 3 and 9 of Table 21, to test the effects of drying speed on the coating appearance and morphology. The drug content transfer efficiency of the coating solution was determined to be over 90 percent. The small transferred amounts in line 2 gave the better coating morphology in that the coating membrane is clear, most transparent and even on the slides. When larger amounts of the coating emulsion were transferred to the slides, lines 3 and 9, the coating became slightly opaque. The results suggested that it may be beneficial in the coating of slides and balloons that multiple passes be utilized to achieve the best coating morphology and appearances.

In a fifth experiment, an aqueous coating solution using PEG 400 and BHT as the solubility and transport enhancers was formulated. To a tared 10-ml scintillation vial was added about 100.5 mg of sirolimus (rapamycin, stock #124623500 batch #RB5070), followed by about 10.1 mg of PEG 400 (Aldrich), and 9.9 mg of BHT (Aldrich). One point five ml (1.5 ml) of acetone was then added to dissolve the above components under shaking. Once the solution became completely clear, 0.5-ml of water was slowly added to the solution. The mixed solution remained a clear and stable solution at room temperature. The composition of the coating formulation is shown in Table 24.

TABLE 24
Aqueous coating formulation using PEG 400, BHT (C1)

| | | Actual am |
|---|---|---|
| | Formulation | in 2 mL |
| | C1 | solution |
| Sirolimus conc | 50 | 100.5 |
| (mg/ml) | | |
| PEG 1000 | 25 | 10.1 |
| BHT (mg/ml) | 5 | 9.9 |
| Acetone (%) | 75 | 1.5 |
| H2O (%) | 25 | 0.5 |

The clear solution of the formulation of Table 24 was transferred to a glass slide for coating morphology studies. A Gilson pipetteman was used to transfer 50 ul of the coating solution onto a pre-weighed glass slide. The coating spot on the slides was allowed to dry at room temperature in a laminar hood. The coating spots gradually become opaque after drying. The weight of the slides with coated spots were measured and recorded in line 6 of Table 21. A larger amount of coating solution C1 was similarly transferred to a glass slide with various amounts, recorded in line 10 Table 21, to test the effects of drying speed on the coating appearance and morphology. The drug content transfer efficiency of the coating solution was determined to be over 95 percent. This experiment shows that a higher percentage of an organic solvent (acetone) resulted in a clear solution as compared to the stable emulsion from the fourth experiment. However, the coated membrane turned out to be hazy and opaque. This morphology is likely due to a faster drying speed with a higher percentage of acetone in the coating solution, 75 percent, compared to the formulation of the fourth experiment wherein the acetone percentage was 60 percent. The slightly lower acetone concentration led to a slower drying process and a more even and transparent appearance.

In a sixth experiment, an aqueous coating solution using PEG 400, BHT, and PVA as the solubility and transport enhancers was formulated. To a tared 10-ml scintillation vial was added about 100.1 mg of sirolimus (rapamycin, stock #124623500 batch #RB5070), followed by about 10.1 mg of PEG 400 (Aldrich), and 9.9 mg of BHT (Aldrich) and 9.7 poly(vinyl alcohol) (PVA, 80% hydrolyzed from Aldrich). One point five ml (1.5 ml) of acetone was then added to dissolve the above components under shaking. Once the solution became completely clear, 0.5-ml of water was slowly added to the solution. The mixed solution remained a clear and stable solution at room temperature. The composition of the coating formulation is shown Table 25.

TABLE 25
Aqueous coating formulation using PEG 400, BHT, PVA (C2)

| | | Actual am |
|---|---|---|
| | Formulation | in 2 mL |
| | C2 | solution |
| Sirolimus conc | 50 | 100.1 |
| (mg/ml) | | |
| PEG 400 | 25 | 10.1 |
| BHT (mg/ml) | 5 | 9.9 |
| PVA (mg/ml) | 5 | 9.7 |
| Acetone (%) | 75 | 1.5 |
| H2O (%) | 25 | 0.5 |

About 100 ul of the clear solution was transferred to a glass slide to form a membrane. The membrane had a weight of 4.8 mg (96 percent transfer efficiency) and formed a smooth and even film. Furthermore, a 3.0×20 mm PTCA balloon was dipped into the coating solution for ten seconds before being pulled out to dry in the laminar hood. The dried weight of the drug coatings are listed in Table 26. The coating appeared to be translucent to clear. The second dip with about five second duration increased the weight by another 2.6 mg and the coating become thicker and more opaque.

TABLE 26
Drug coating weight on balloon surface after dipping coating

| | Tare | wt w/1 | Net 1 |
|---|---|---|---|
| | weight (g) | coat (g) | coat (g) |
| balloon 1 | 0.0139 | 0.0169 | 0.003 |
| balloon 2 | 0.0159 | 0.0188 | 0.0029 |
| balloon 3 | 0.0471 | 0.0511 | 0.004 |

The coated balloons were then immersed in deionized water (DI water) for two minutes under gentle agitation. The balloons then were clipped to a clamp and placed in a laminar hood to dry for thirty minutes. The coating on the balloons became opaque with a white film on the balloon. On average, the coating lost about 14-54 percent drug coating. The results are listed below in Table 27.

TABLE 27
Loss of coating weight after immersion in water

| | wt | | | | |
|---|---|---|---|---|---|
| | after 1 | wt post water | wt removed | total coat | % |
| | coat (g) | soak (g) | (g) | (g) | removal |
| balloon 1 | 0.0169 | 0.0158 | 0.0011 | 0.0077 | 14.3 |
| balloon 2 | 0.0188 | 0.0165 | 0.0023 | 0.0042 | 54.8 |
| balloon 3 | 0.0511 | 0.0488 | 0.0023 | 0.0077 | 29.9 |

In a seventh experiment, an aqueous coating solution using PEG 400, BHT, PVA and Brij 35 as the solubility and transport enhancers was formulated. To a tared 10-ml scintillation vial was added about 100.0 mg of sirolimus (rapamycin, stock #124623500 batch #RB5070), followed by about 10.1 mg of PEG 400 (Aldrich), and 9.9 mg of BHT (Aldrich) and 10.1 poly(vinyl alcohol) (PVA, 80 percent hydrolyzed from Aldrich), and 5.7 mg of Brij 35 (Polyoxyethyleneglycol dodecyl ether, a nonionic surfactant, Aldrich). One point five ml (1.2 ml) of acetone was then added to dissolve the above components under shaking. Once the solution became completely clear, 0.8-ml of water was slowly added to the solution. The mixed solution remained a clear and stable solution at room temperature. The composition of the coating formulation is shown in Table 28.

TABLE 28
Aqueous coating formulation using PEG 400, BHT, PVA (B2)

| | | Actual am |
|---|---|---|
| | Formulation | in 2 mL |
| | B2 | solution |
| Sirolimus conc | 50 | 100.0 |
| (mg/ml) | | |
| PEG 400 | 25 | 10.1 |
| BHT (mg/ml) | 5 | 9.9 |
| PVA (mg/ml) | 5 | 10.1 |
| Brij 35 (mg/ml) | 2.5 | 5.7 |
| Acetone (%) | 60 | 1.2 |
| H2O (%) | 40 | 0.8 |

This coating solution was clear, in contrast to the stable emulsion of B1 from the fourth experiment. This is possibly caused by the addition of PVA and Brij 35 which helps the solubility of sirolimus in the mixed solution. About 100 ul of the clear solution was transferred to a glass slide to form a membrane. The membrane had a weight of 4.6 mg (92 percent transfer efficiency) and formed a smooth and even film. Furthermore, a 3.0×20 mm PTCA balloon was dipped into the coating solution for 10 seconds before being pulled out to dry in the laminar hood. The dried weight of the drug coating was 2.2 mg. The coating appeared to be translucent to clear. The second dip increased the weight by another 3.0 mg and the coating become more opaque. The third dip increased the coating weight by another 3 mg. Also the speed of the dipping is critical in that prolonged exposure to the coating solution will dissolve the previously laid down coating there. The coating weight after each dipping step and final coating weight were listed in Table 29.

TABLE 29
Drug coating weight on balloon surface after dipping coating

| | tare weight | wt w/1 | net 1 | wt w/2 | net 2 | wt w/3 | net 3 | total coat |
|---|---|---|---|---|---|---|---|---|
| | (g) | coat (g) | coat (g) | coat (g) | coat (g) | coat (g) | coat (g) | wt (g) |
| balloon 1 | 0.0234 | 0.029 | 0.0056 | 0.0308 | 0.0018 | 0.0311 | 0.0003 | 0.0077 |
| balloon 2 | 0.018 | 0.019 | 0.001 | 0.0196 | 0.0006 | 0.0222 | 0.0026 | 0.0042 |
| balloon 3 | 0.0231 | 0.0255 | 0.0024 | 0.0276 | 0.0021 | 0.0308 | 0.0032 | 0.0077 |

From the study it appears that between 4-7 mg of coating was added to the balloon surface after three dipping steps. The coating appeared to be clear to translucent.

In the final step of the study, the coating balloons were then immersed in deionized water (DI water) for two minutes under gentle agitation. The balloons then clamped to a clip and were placed in a laminar hood to dry for thirty minutes. The coating on the balloons became an opaque and white film on the balloon. On average, the coating lost about 70 percent weight as shown in the Table 30.

TABLE 30
Loss of coating weight after immersion in water

| | wt | | | | |
|---|---|---|---|---|---|
| | after 3 | wt post water | wt removed | total coat | % |
| | coat (g) | soak (g) | (g) | (g) | removal |
| balloon 1 | 0.0311 | 0.0257 | 0.0054 | 0.0077 | 70.1 |
| balloon 2 | 0.0222 | 0.0192 | 0.003 | 0.0042 | 71.4 |
| balloon 3 | 0.0308 | 0.0256 | 0.0052 | 0.0077 | 67.5 |

The loss of coating was probably further facilitated by the additional use of Brij 35 (surfactant) and PVA (water soluble polymer) which hydrate upon contact with water. The amount of Brij 35 and PVA in the final formulation may be adjusted to control the percent of drug release from the balloon surface.

Some of the above listed aqueous formulations are suitable for use as a PTCA balloon surface coating, especially exemplified by formulations B1, B2, C1, and C2. The various excipients may be adjusted to control the coating solution for better stability and ease of detachment from the balloon surface upon deployment.

The formulations, B1 and C1 as listed in Table 21, wherein a good balance of organic solvent such as acetone and water is reached, together with the optional use of excipients such as PEG, PVA and BHT may be used to control separation of the drug coating from the balloon surface. These excipients, by their amphiphilic nature (PEG, Brij 35, and PVA) should also facilitate the transport of drug into the tissue and enhance their tissue retention as well. An additional detachment facilitating agent such as PVA and non-ionic surfactant (Brij 35) as used in the formulation set forth in Table 25 for C2, and Table 26 for B2 also helped separate the drug coating from the balloon surface.

Accordingly, Table 31 below lists the preferred formulation ranges for surface coatings based upon the individual formulations B1, B2, C1 and C2 described above.

TABLE 31
Formulation summary

| | B1 | C1 | B2 | C2 |
|---|---|---|---|---|
| Sirolimus conc | 50 | 50 | 50 | 50 |
| (mg/ml) | | | | |
| PEG 400 (mg/ml) | 5 | 5 | 5 | 5 |
| BHT (mg/ml) | 5 | 5 | 5 | 5 |
| Brij 35 (mg/ml) | N/A | N/A | 2.5 | 2.5 |
| Acetone/H2O | 60/40 | 75/25 | 60/40 | 75/25 |

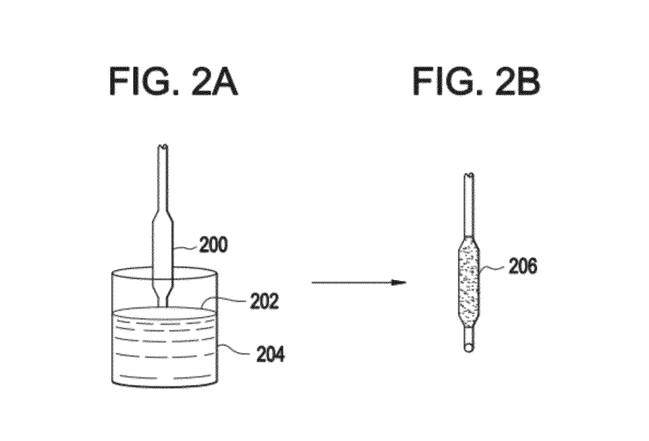

It is important to note that the balloon or other medical device may be coated in any suitable manner. For example, the balloon may be spray coated, have the coating brushed or wiped on, or dip coated. FIG. 2A illustrates a balloon 200 being dipped into a coating solution, suspension and/or emulsion 202 contained within a vial 204 and FIG. 2B illustrates the coated balloon 206. This process, as described herein, may be repeated multiple times to achieve the desired drug concentration.

It is important to note that when utilizing a balloon or other expandable member to deliver drugs and/or therapeutic agents, the balloon or other expandable member is expanded to a diameter at least ten percent higher than the nominal diameter of the vessel. This over expansion serves a number of functions, including facilitation of the drug and/or therapeutic agent into the surrounding tissues. Furthermore, the level and duration of inflation or expansion may influence the extent of drug uptake in the target tissue.

Another formulation of a rapamycin may be specifically tailored for balloon delivery. More specifically, a formulation of a rapamycin designed for release from the surface of a balloon or other expandable device for a very short period of time is disclosed. Important requirements for a drug coated device to show sufficient efficacy include having an active pharmaceutical ingredient (API) selected to treat restenosis properly coated onto the surface of an implantable medical device, particularly a PTCA balloon, in a sufficient quantity, and to be released at the site of intervention in sufficient quantity within a short period of time when the device surface is in contact with the lesion. A number of compositions and coating methods have been proposed to achieve a formulation that is potent enough to treat lesions such as a de novo stenosis in the coronary artery or a restenosis following an angioplasty procedure, for example, in-stent restenosis. The main challenges of devising such a formulation lie in the multiple technical requirements of making the drug formulations such that they adhere to the balloon surface until the time for delivery into the tissue, keeping the coating stable during storage and the transit through the vasculature to the site of intervention, and having the coating released in sufficient quantities upon deployment. These requirements usually require more than one excipient or sets of excipients that have properties that may be exploited for opposing purposes. For instance, excipients may be required to enhance the adhesion of the coating formulations to the balloon surface or the surface in the balloon folds so that the API in the coating is not lost upon expansion. On the other hand, excipients may be needed to facilitate the detachment of the API from the surface and enter the arterial tissue for its intended anti-restenotic and/or anti-proliferative functions. These two requirements are often contradicting in nature and experimentation is required to fine-tune or balance these opposing requirements in the final formulation.

During experimentation to determine formulations, it was observed that butylated hydroxytoluene, (BHT), seemed to be effective in enhancing the adhesion of the sirolimus, a rapamycin that has shown remarkable efficacy when used as the API in drug eluting stents, to the surface of the device or balloon. Several methods of evaluating the adherence of the sirolimus coating to the balloon surface and the final percent delivery of the sirolimus at the lesion site seems to suggest that BHT in a certain ratio to sirolimus (0.5 to 5 percent w/w) is effective in enhancing the adhesion and retention of the rapamycin coating to the surface of the balloon during adhesion testing. In addition, the porcine studies detailed herein also suggest that the rapamycin coating on a PTCA balloon with 5 percent BHT admixed in the sirolimus coating formulation was effective in suppressing intimal hyperplasia in a standard porcine coronary artery intimal proliferation model as compared to uncoated controls.

A number of experiments were conducted to determine the formulations that achieved the minimal requirements set forth above. While the exact mechanism for the enhancement of the sirolimus formulation via the use of BHT to the balloon surface and its ultimate enhanced antiproliferative efficacy is not completely understood, it is reasonable to assume that it either enhanced the adhesion of the rapamycin to the balloon surface, or made the final formulation more compliant thereby allowing the formulation or coating to remain on the balloon surface more securely, while enhancing the release of the rapamycin coating at the lesion site due to its more hydrophilic nature. Accordingly, BHT in this particular application, may have multiple roles.

In accordance with a set of typical balloon coating formulations, rapamycin is dissolved in a solvent system that has multiple organic solvents such as ethanol, acetone, or isopropanol (IPA) mixed with water in a preselected ratio. A typical ratio between organic solvent to water was 3.4/1 (volume/volume). The drug and BHT were added to the organic solvent for full dissolution before water was added to make the final coating formulation. The target concentration of sirolimus in the coating formulation is designed based on the calculation that the final surface density of sirolimus on the balloon surface should be up to about 7 μg/mm² of the balloon surface, although the final rapamycin concentration or density on the surface as determined by analytical method such as high pressure liquid chromatography (HPLC) was lower than the target concentration. The balloon catheter used in the present formulation and porcine studies has a diameter of 3.5 mm and a length of 20 mm and a total nominal surface area of 220 square millimeters. Balloons meeting this description are commercially available from Cordis Corporation and sold under the name FIRE STAR® PTCA balloon (3.5×20 mm). The final target sirolimus concentration in the coating is around 1.54 mg/balloon. These balloons are mounted with a standard bare metal stent such as the Bx VELOCITY Coronary Stent or any newer generation coronary and/or peripheral stent available from Cordis Corporation. During experimentation, it was also observed that in the acetone/ethanol/water solvent system FIRE STAR® PTCA balloon with a hydrophilic coating is not as conducive to a durable drug coating when compared to a comparable Fire Star® PTCA balloon without a hydrophilic surface treatment prior to the application of the sirolimus drug coating. Drug coating on a hydrophilic balloon surface lost substantially more drug during the coating adherence tests. This observation is not surprising in that the hydrophilic treatment is designed to decrease the tackiness of the surface. Accordingly, a drug coating formulation should preferably be applied to an unmodified balloon surface.

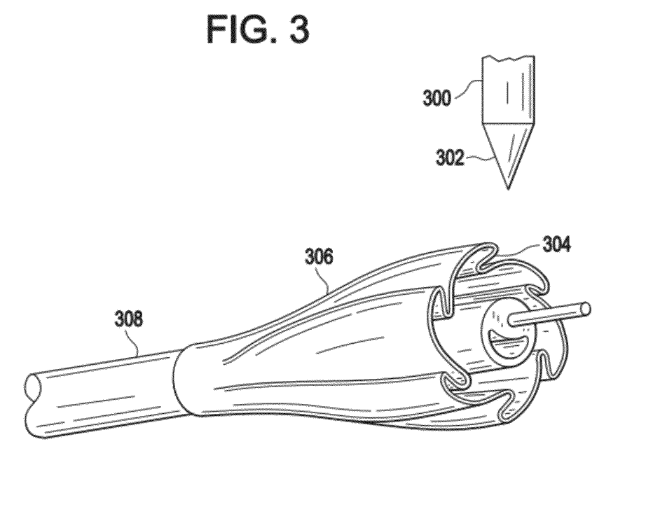

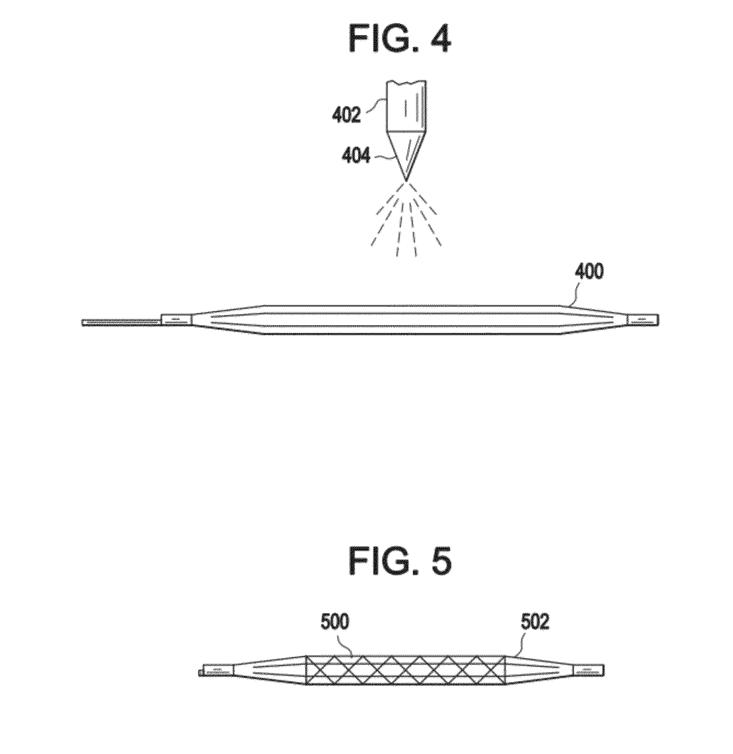

In accordance with a first experiment, multiple balloon coating formulations of sirolimus with BHT at 0 percent, 1 percent, and 5 percent (w/w) were prepared. To a vial containing 3.4 ml of IPA were added 220 mg of sirolimus and 2.2 mg of BHT (1 percent BHT formulation). Upon agitation and full dissolution of sirolimus and BHT in the solvent, 1 ml of water was added and agitated to form the final coating formulation. The concentration of sirolimus in the final coating formulation was 50 mg/ml. The formulations with BHT at 0 percent and 5 percent (11 mg) were similarly prepared. The sirolimus coating solutions (16 ul) were pipetted to the folds of a folded FIRE STAR® PTCA balloon and dried at room temperature. FIG. 3 illustrates the use of a pipette 300 to precisely deliver the sirolimus formulation 302 into the folds 304 of a balloon 306 on the end of a delivery catheter 308. A second application of each formulation was applied to the balloon surface utilizing an identical procedure and dried to complete the coating process. It is important to note that any number of processes may be utilized to coat the balloon. For example, the balloon may be dip coated as described above or have the formulation sprayed onto the surface of a balloon 400 as illustrated in FIG. 4. In this process, a spray head 402 is utilized to deliver the formulation 404 onto the surface of the balloon 400. In addition, various syringe pumps and/or micro dispensers may be utilized to coat the balloon surface or the surfaces of the balloon folds. Also, the balloon may be entirely coated or just certain regions such as the balloon folds.

The coated FIRE STAR® PCTA balloons were then tested in a wet-adhesion test that simulates the deployment procedure of a drug coated balloon. The sirolimus loss test consisted of passage of the drug coated balloon through a standard hemostatic valve, then a guiding catheter (Medtronic Launcher® Catheter JL 3.5 6 French available from Medtronic Corporation), and one minute incubation in stirred blood (37 degrees C.). The amount of sirolimus remaining in the balloon after the incubation is assayed by HPLC to arrive at the percentage of sirolimus loss during the test. The results of the drug loss test for each formulation is given in Table 32.

TABLE 32
Loss of sirolimus coating with varying concentrations
of BHT in the coating formulation

| Balloon with | | | |
|---|---|---|---|
| Hydrophilic | Solvent | BHT/sirolimus | Sirolimus Loss in |
| treatment | system | (% w/w) | test (%) |
| Yes | Acetone/ | 0% | 78 ± 5 |
| Yes | ethanol/water | 1% | 76 ± 3 |
| Yes | | 5% | 40 ± 13 |
| No | Acetone/ | 0% | 49 ± 3 |
| No | ethanol/water | 1% | 49 ± 4 |
| No | | 5% | 33 ± 5 |
| Yes | IPA/water | no | 22 ± 7 |
| Yes | | 1% | 21 ± 1 |
| Yes | | 5% | 2 ± 5 |

The test results in Table 32 clearly demonstrate that a sirolimus solution comprising 5 percent BHT is effective in reducing the loss of sirolimus during the simulated deployment procedure. The data also suggested that in the acetone/ethanol/water solvent system a hydrophilic treatment on the PTCA balloon adversely affects the retention or adhesion of sirolimus on the balloon surface. The sirolimus solution comprising 5 percent BHT was determined to be a preferred formulation and further used in the porcine tests of its efficacy in a standard porcine injury and restenosis model, details of which are given subsequently.

In accordance with a second experiment, the efficacy of a PTCA balloon coated with the 5 percent BHT solution was tested in a porcine injury model. The balloon coating formulation of sirolimus and BHT (5 percent BHT, w/w) was made according to the procedure described above. In total, three coating solutions of sirolimus and BHT (5 percent BHT, w/w) and one coating solution without BHT were prepared for the study. A standard CYPHER® Sirolimus-eluting Coronary Stent available from the Cordis Corporation was used as a control for the study. Both FIRE STAR® PTCA balloons (3.5 mm×20 mm, with total surface area of 220 mm²) with hydrophilic treatment and the ones without a hydrophilic treatment were tested in the study. The four formulation compositions are set forth in Table 33 below. The final coating density of sirolimus and sirolimus loss during expansion were measured by HPLC. The tissue concentration in the porcine coronary arteries was measured by liquid chromatography-mass spectroscopy (LC-MS). The amount of intimal hyperplasia was determined by standard quantitative coronary angiography (QCA) at day 30.

TABLE 33
sirolimus coating formulations tested in porcine
intimal hyperplasia model studies

| Hydrophilic | | Sirolimus conc in | |
|---|---|---|---|
| coat on | Solvent system | coating solution | BHT/sirolimus |
| balloon | (v/v) | (mg/ml) | (%, w/w) |
| yes | IPA/Water | 50 | 0 |
| yes | (3.4/1) | 50 | 5 |
| no | | 50 | 5 |
| no | Acetone/ethanol/ | 50 | 5 |
| | water (50/40/10) | | |

Specifically, 2.5 ml of each coating solution was prepared and two applications of 16 μl coating solution was applied to the PTCA balloon surface and dried before use as described above. The percentage of drug coating loss after expansion in air (dry state) and post deployment in the coronary artery of a pig are shown below in Table 34.

TABLE 34
Sirolimus coating loss post expansion

| Hydrophilic | | | | | |
|---|---|---|---|---|---|
| coat | | BHT/sirolimus | Coating apperance | coating loss during | coating retention |
| on balloon | Solvent system (v/v) | (%, w/w) | before EO | dry expansion (%) | post deploy (%) |
| yes | IPA/Water (3.4/1) | 0 | white, homogeneous, | 63.8 ± 7.2 | 3.2 |
| | | | somewhat loose coating | | |
| yes | | 5 | white, homogeneous, | 66.6 ± 10.8 | 3 |
| | | | somewhat loose coating | | |
| No | | 5 | only slightly white, | 43.3 ± 5.1 | 14.7 |
| | | | almost homogeneous | | |
| No | Acetone/ethanol/ | 5 | slightly white, spotty, | 40.3 ± 2.2 | 11.1 |
| | water (50/40/10) | | stripes, folds loosened | | |

From the data in Table 34 it is clear that the hydrophilic coating or treatment on the PTCA balloon prior to sirolimus formulation coating did cause more drug loss in drug coating during dry state expansion and consequently resulted in less drug retention in the coating post deployment. This is not surprising in that a hydrophilic coating is designed to decrease the tackiness of the surface and possibly repel subsequent coatings and facilitate the coating detachment from the hydrophilic coating after deployment. The two coating formulations put on the balloon surface without a prior hydrophilic treatment resulted in less loss of drug coating during dry state expansion and retained more drug on the balloon after deployment.

From the data presented in Table 35 shown below, it is clear that for the two groups that had a hydrophilic coating before the sirolimus coating was applied, the addition of 5 percent BHT to the coating formulation did result in higher initial tissue concentrations.

TABLE 35
Sirolimus tissue concentration at various times post-implantation

| | | | | Sirolimus conc in artery tissue post-deploy (ng | | | |
|---|---|---|---|---|---|---|---|
| Hydrophilic | | Sirolimus conc | BHT/sirolimus | sirolimus/mg tissue) | | | |
| coat on balloon | Solvent system (v/v) | (mg/ml) | (%, w/w) | 20 min | 24 hr | 8 day | 30 day |
| yes | IPA/Water (3.4/1) | 50 | 0 | 219 ± 85 | 16 ± 11 | 16 ± 18 | 3.2 ± 2.8 |
| yes | | 50 | 5 | 313 ± 61 | 40.7 ± 14.6 | 9.8 ± 10.4 | 8.4 ± 5.7 |
| No | | 50 | 5 | 218 ± 96 | 39 ± 37 | 14 ± 15 | 5.0 ± 4.8 |
| No | Acetone/ethanol/ | 50 | 5 | 382 ± 190 | 25 ± 20 | 21 ± 36 | 12 ± 18 |
| | water (50/40/10) | | | | | | |

For the two groups that used balloons with prior hydrophilic treatment before sirolimus and BHT 5 percent coating, there seemed to be a higher initial tissue concentration for the acetone/ethanol group, presumably tied to the different physical state of the coating during the expansion. The slightly lower initial tissue concentration of sirolimus correlated in IPA/water group correlated to the slightly lower amount of sirolimus remaining on the balloon surface post deployment. Regardless of the formulation, the tissue concentration of sirolimus at 20 minutes, 24 hours, 8 days and 30 days were all above therapeutic efficacious levels shown in a comparable drug eluting stent, generally in the range of 1 ng sirolimus/mg of tissue.

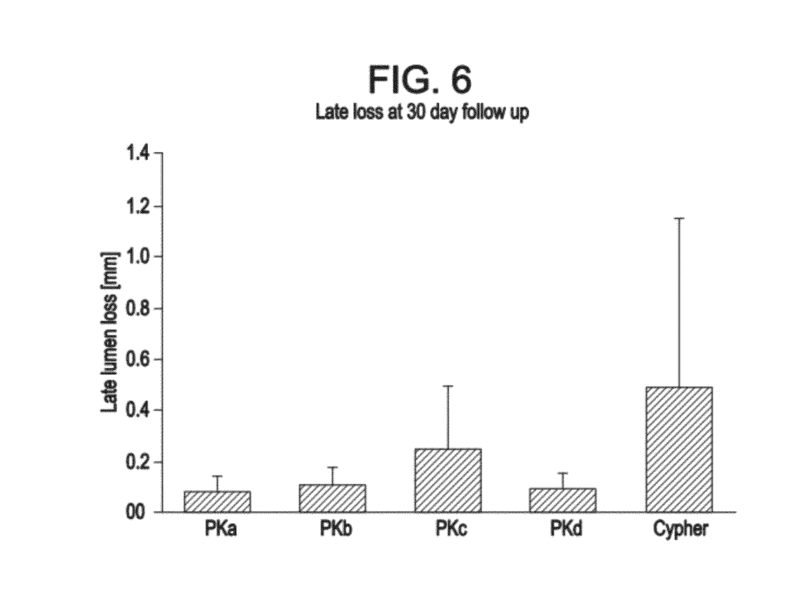

The sirolimus and BHT coated balloons and the control CYPHER® Sirolimus-eluting Coronary Stents were used in a standard porcine coronary artery implantation study. The over-sizing of the balloon during balloon expansion in the study was controlled at 10-20 percent. The end point is late lumen loss at 30 days post implant using QCA. The codes and formulations for the four sirolimus coated balloons and CYPHER® Sirolimus-eluting Coronary Stents control in the 30 day PK studies are listed below in Table 36 and the 30-day late lumen loss of the different groups is illustrated graphically in FIG. 6.

TABLE 36
Formulations used in porcine 30 day implantation studies

| Hydrophilic | | Sirolimus | | Porcine |
|---|---|---|---|---|
| coat on | Solvent system | conc | BHT/sirolimus | study |
| balloon | (v/v) | (mg/ml) | (%, w/w) | code |
| Yes | IPA/Water | 50 | 0 | PKc |
| Yes | (3.4/1) | 50 | 5 | Pka |
| No | | 50 | 5 | PKb |
| No | Acetone/ethanol/ | 50 | 5 | PKd |
| | water | | | |
| | (50/40/10) | | | |
| Cypher | N/A | N/A | N/A | Pkcy |

The study results demonstrated that all four formulations had similar late loss (mm) comparable to the clinically proven CYPHER® Sirolimus-eluting Coronary Stent control.

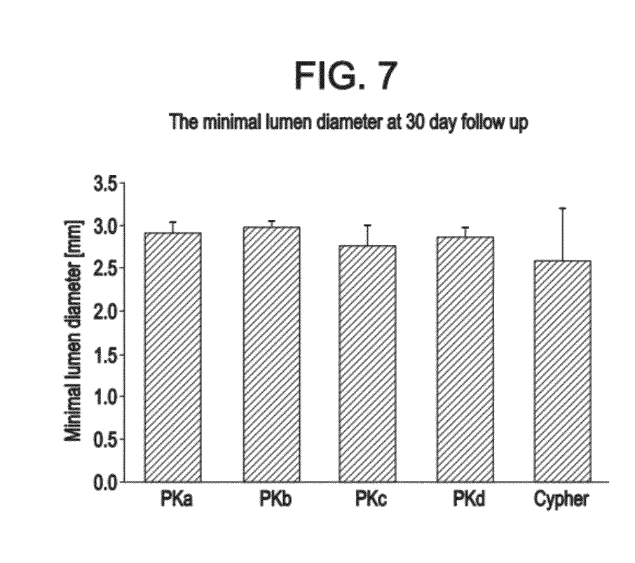

Similar measurements of efficacy such as the minimal lumen diameter at 30 days also suggested that sirolimus coated balloons had comparable efficacy as the CYPHER® Sirolimus-eluting Coronary Stent group in the study as graphically illustrated in FIG. 7.

It may be beneficial to utilize a bare metal stent in conjunction with a drug coated balloon to further decease the chance of vessel closure. In addition, the placement of the bare metal stent over the drug coated balloon for delivery thereof may also serve to protect the drug coating on the balloon surface or in the folds. FIG. 5 illustrates a stent 500 on a drug coated balloon 502.

In accordance with an exemplary embodiment, the present invention is directed to creating a non-aqueous liquid formulation of a paclitaxel composition comprising paclitaxel, an antioxidant, a film-enhancing agent and/or film-forming agent, and at least one volatile, non-aqueous solvent. The formulation is preferably affixed to the surface of a medical device by any suitable means and dried such that substantially no residual solvent remains. As used herein, the term non-aqueous shall mean an organic solvent other than water, the term film-enhancing agent shall mean a naturally derived or synthetic material that enhances the formation of a coating or film, wherein the normal range for the inclusion of such an agent is between about 0.01 percent (wt/wt) to about 20.0 percent (wt/wt), and the term volatile shall refer to a material and/or solvent with a boiling point of below 150 degrees C. at one (1) atmosphere. The paclitaxel composition may be utilized as a coating on an expandable medical device, for example, a balloon, such that the expansion of the device facilitates the contact between the coating and tissue, and the uptake of the liquid formulation into the tissue comprising the vessel walls in which the device is utilized.

A number of experiments as set forth herein suggest that paclitaxel as well as sirolimus elicited efficacious anti-restenostic and anti-inflammatory responses in a porcine coronary implant model. These above-described experiments also showed that these formulations generally had substantial loss of the coating, both during the coating, folding and packaging process, and during transit to the deployment site in the vasculature. Thus, there exists a need to further enhance the adhesion of the paclitaxel formulations to the balloon surface to minimize the loss of the active pharmaceutical agent; namely, paclitaxel. Accordingly, a series of non-aqueous formulations were created and coated onto glass slides and balloon catheters to demonstrate the enhanced adhesion of a drug coating to a balloon surface by utilizing a film-forming and/or film-enhancing agent as part of the composition.

Non-aqueous formulations or compositions offer a number of advantages over aqueous formulations or compositions. As compared to non-aqueous formulations, aqueous formulations require longer processing time in that they take longer to dry. In addition, non-aqueous formulations are less stable than their non-aqueous counterparts. The desired characteristics for a composition to be utilized on an expandable device such as a balloon include good coating adhesion, good release kinetics, good film forming properties and drug or therapeutic agent stability. In the exemplary embodiment described herein, the antioxidant (e.g. BHT) functions to promote the adhesion of the final formulation to the device, stabilizes the therapeutic agent, and functions to facilitate favorable release kinetics by disrupting the crystallinity of the therapeutic agent thereby promoting release from the device surface and tissue uptake. In the exemplary embodiment described herein, the film forming agent (e.g. PVP) functions to promote better adhesion of the final composition to the surface of the device thereby serving to prevent premature release of the therapeutic agent from the device during preparation and delivery. In addition, both the antioxidant and the film-forming agent function to increase transport of the therapeutic agent from the device and into the surrounding tissue.

The following experiments serve to illustrate the principles and formulations briefly described above. Many of the excipients may be interchanged to enhance one aspect or another of the formulations, without affecting the efficacy of the particular formulation. A complete listing of these excipients is given subsequently.

In a first set of experiments in accordance with the present invention, a series of ethanol solutions containing paclitaxel (PTX, also known as TAXOL® as a commercial dosage form), butylated hydroxyl toluene (BHT), and K90 and K30 (polyvinyl pyrrolidone), a PVP from BASF) were prepared. K90 is a specific grade of PVP from BASF with a K value of 80-100, and a high molecular weight (Mn) of about 360 KD according to the manufacturer. The composition of the PTX coating solutions containing one percent K90, one percent K30 and a control composition containing no PVP is shown in Table 37.

TABLE 37
Ethanol solutions of paclitaxel, BHT, and K30 or K90

| Code | PVP, mg | BHT, mg | PTX, mg | ethanol, ml |
|---|---|---|---|---|
| PXB-0 (control) | 0 | 1.3 | 25 | 1 |
| PXBK30-1 (K30) | 0.25 | 1.3 | 25 | 1 |
| PXBK90-1 (K90) | 0.25 | 1.3 | 26 | 1 |

Specifically, about 25 mg of paclitaxel was added to two tared 10-ml scintillation vials (Calbiochem, Cat #580555, Lot #D00077065), followed by about 1.3 mg of BHT (Lot #K36760774 from EMD), and 1 ml of ethanol (Catalog #: EX0278-6, lot #:50043, from EMD). The two scintillation vials were then capped tightly and the solid solvent mixtures were agitated with a lab vortex mixer for about thirty seconds before being placed in a ventilation hood. The vials were agitated by the vortexer several times until the drug and BHT were fully dissolved to form homogeneous solutions.

Separately a K90 stock solution (1 mg/ml) in ethanol was prepared by weighing 10 mg of K90 into a tared scintillation vial followed by 10 ml of ethanol. The vial was then capped tightly and vortexed and placed in a ventilation hood until the K90 was fully dissolved. An aliquot of 250 μl of the stock solution was then added to the above paclitaxel/BHT solution (PXBK90-1) to make the final K90 concentration at one percent (wt/wt) relative to paclitaxel. The final composition of the one percent K90 containing coating solution is shown in Table 37. The control vial did not contain any K90.

A PTX/BHT coating solution containing one percent K30 was made in parallel. The procedure was similar to that used in making the one percent K90 solution. A K30 stock solution (1 mg/ml) in ethanol was prepared by weighing 10 mg of K30 into a tared scintillation vial followed by 10 ml of ethanol. The vial was then capped tightly and vortexed and placed in a ventilation hood until the K30 was fully dissolved. An aliquot of 250 μl of the stock solution was then added to the above paclitaxel/BHT solution (PXBK30-1) to make the final K30 concentration at one percent (wt/wt) relative to paclitaxel. The control vial did not contain any K30. The final composition of the one percent K30 containing coating solution is shown in Table 37, as well as the control coating solution.

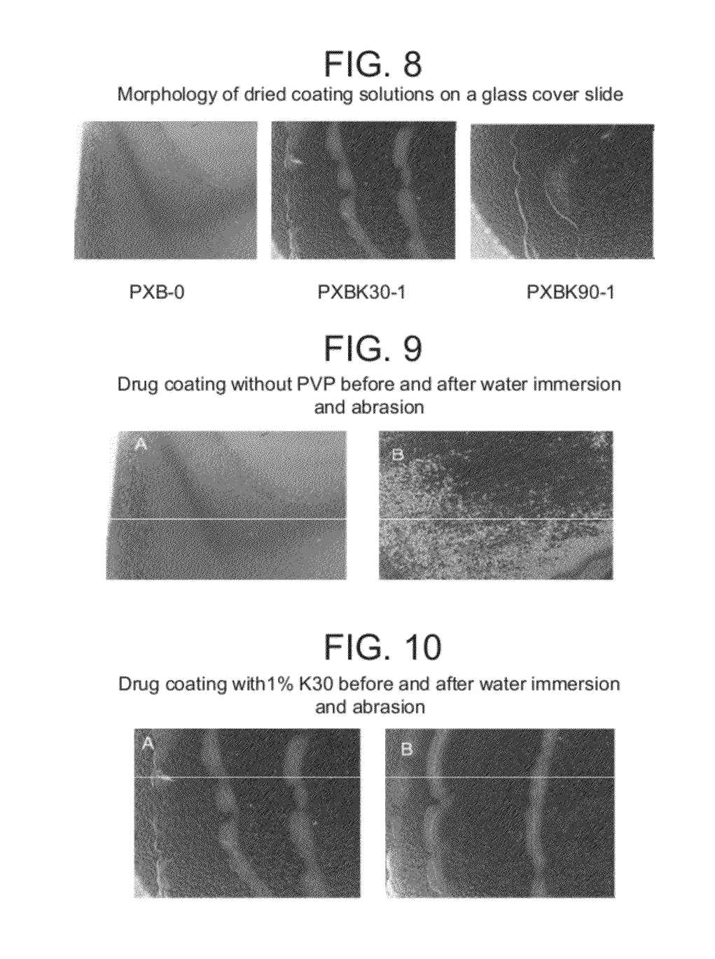

Once the three coating solutions were made, they were deposited onto three individual regular glass cover slides with a 25 μl increment using a calibrated Eppendorf pipette and dried in a ventilation hood at room temperature. To achieve the desired coating thickness and density, and observe coating morphology changes, up to three depositions of coating solutions were made onto the slides. The coated slides were then air dried overnight in a ventilation hood. The morphology of each dried coating on the glass cover slide was captured by a Keyence microscope fitted with a digital optical camera. The images are illustrated in FIG. 8.

The images illustrated in FIG. 8 show that the addition of either K30 and K90 at the one percent level was sufficient to make the dried coating transparent, compared to the control coating (PXB-0) on the left side of FIG. 8, which was an opaque white and powdery mass on the glass slide. The three rings of the coatings in the images denote the three depositions of the coating solutions on the glass slides. These rings suggest that fast evaporation of the coating solutions prevent them from fully dissolving the coatings previously laid down on the slides. Nonetheless both films (PXBK30-1 and PXBK90-1) were transparent and adhering well to the glass.

To evaluate the adhesion of the above films to the glass, the coated slides were immersed in deionized water (DI water) for five minutes at room temperature. The slides were then dried and subjected to gentle axial abrasion using a lint-free lab Kimwipe paper towel. The images of the coating before and after water immersion and abrasion are shown in FIGS. 9 to 11.

Image B of FIG. 9 illustrates the loss of coating across the whole coated area on the slide compared to the intact coating in Image A, suggesting that water immersion and gentle wiping abrasion was sufficient to loosen and remove part of the coating from the glass surface.

Image B of FIG. 10 illustrated no appreciable loss of coating occurred across the whole coated area on the slide compared to the intact coating in Image A, suggesting that water immersion and gentle wiping abrasion was not able to remove the bulk of the coating from the glass surface. The slightly less transparent image in Image B suggests that water was able to penetrate into the coating and remove a portion of the water soluble K30 and possibly BHT during the immersion. The coating integrity might be affected as well.

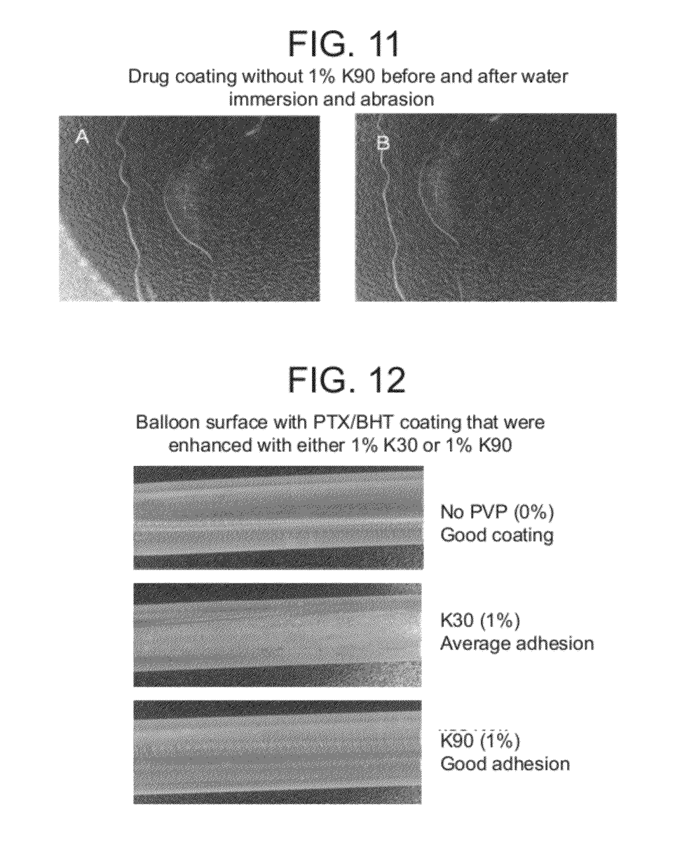

Image B of FIG. 11 illustrates no appreciable loss of coating across the whole coated area on the slide compared to the intact coating in Image A, suggesting that water immersion and gentle wiping abrasion was not able to remove any of the coating from the glass surface. The intact coating in Image B after water immersion and Kimwipe abrasion suggested that water was not able to penetrate into the coating to alter the appearance or the integrity of the coating. The enhancement of coating adhesion to the glass and coating integrity with the addition of K90 was more effective than K30 as illustrated in FIG. 10. This is not surprising considering the much higher Mn of K90 as compared to K30. The much higher Mn makes K90 a better film forming polymer.

After the preliminary evaluation of coating morphology and adhesion of K30 and K90 enhanced film on glass slides, a series of coating studies were performed on standard PTCA balloons. The balloons were inflated with an endoflator to a pressure of about two atmospheres and cleaned with ethanol-soaked lint-free Kimwipe. The cleaned balloon was air dried for two minutes before coating with various paclitaxel/BHT/PVP coating solutions. A coating solution was deposited onto the surface of an inflated balloon (two atmospheres) while the balloon was gradually rotated. Up to three applications of coating were deposited with an Eppendorf pipette. Two minutes drying time was allowed between coating applications. The final coated balloons were air-dried in ventilation hood overnight before evaluation with a Keyence microscope equipped with a digital camera. The coated balloons after inflation to a pressure of about three atmospheres are shown in FIG. 12.

The images in FIG. 12 show that all balloon coatings have good coating appearances. This is probably due to the fact that a relatively small amount of coating was applied to the balloon surface. The next steps would require a much higher coating concentration that leads to a pharmaceutically efficacious range of up to 10 μg/mm² of balloon surface. Nonetheless, the K90 containing film appeared to show a more uniform and streak-free coating on the balloon.

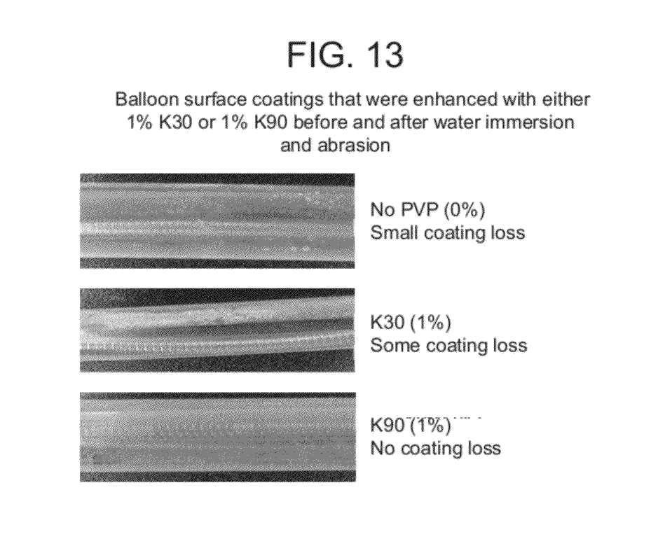

These balloon coatings were then subjected to a five minute immersion in water and blotted dry with a Kimwipe. A clean sheet of Kimwipe was then applied axially along the coating on the balloon with a pinching force to mimic the loss of the coating during transit in the blood vessels to a treatment site. The images of the balloons after water immersion and abrasion are illustrated in FIG. 13.

The images in FIG. 13 illustrate that the control coating (top panel, without PVP) lost part of the coating after water immersion and abrasion. The coating with one percent K30 also lost part of the coating, mainly along the middle section of the coating and had a streaky appearance. This is possibly due to the holding pattern of the Kimwipe during the wiping process. The coating with one percent K90, on the other hand, showed no appreciable loss of coating after the abrasion process. This suggests that K90 was the most effective at resisting the water ingress and retaining the adhesion of the film to the balloon surface. The range of K30 and K90 in the coating formulae at around one percent was in part based on previous experiments in which K30 and K90 all showed effective control of coating uniformity and enhancement of adhesion of the coating to the balloon surface.

The above studies illustrate that a biocompatible synthetic water soluble polymer, poly(vinylpyrrolidone), when used at optimal levels in the coating formulation, led to much improved coating appearances and much better resistance to physical abrasion that is similar to the resistance that a coated balloon will likely encounter en route to the treatment site before inflation. The studies also suggest that K90 appeared to be more effective than K30 at forming films and better at enhancing the resistance of the coating to water ingress and abrasion forces. This finding, however, does not necessarily mean that K90 would be more useful than K30 when tested in vivo since ease of detachment of the coating from the balloon was desirable for after coating transfer at the deployment site.

Other pharmaceutic carriers or film-forming and/or film-enhancing agents other than PVP include, hydroxyalkylcelluloses, such as hydroxypropylcellulose and HPMC, hydroxyethyl cellulose, alkylcelluloses such as ethycellulose and methylcellulose, carboxymethylcellulose; sodium carboxymethylcellulose, hydrophilic cellulose derivatives, polyethylene oxide (PEO), polyethylene glycol (PEG); cellulose acetate, cellulose acetate butyrate, cellulose acetate phthalate, cellulose acetate trimellitate, polyvinylacetate phthalate, hydroxypropylmethyl-cellulose phthalate, hydroxypropylmethyl-cellulose acetate succinate; poly(alkyl methacrylate); and poly(vinyl acetate) (PVAc), poly(vinyl alcohols) (PVA), carboxyvinylpolymers, crosslinked polyvinylpyrrolidone, carboxymethyl starch, potassium methacrylate-divinylbenzene copolymer, hydroxypropylcyclodextrin, alpha, beta, gamma cyclodextrin or derivatives and other dextran derivatives, copolymers derived from acrylic or methacrylic acid esters, copolymers of acrylic and methacrylic acid esters.

Examples of other suitable polymer film-forming and/or film-enhancing agents include, either alone or in combination, shellac, glucans, scleroglucans, mannans, xanthans, cellulose, natural gums, seaweed extract, plant exudate, agar, agarose, algin, sodium alginate, potassium alginate, carrageenan, kappa-carrageenan, lambda-carrageenan, fucoidan, furcellaran, laminarin, hypnea, eucheuma, gum arabic, gum ghatti, gum karaya, gum tragacanth, guar gum, locust bean gum, okra gum, quince psyllium, flax seed, arabinogalactin, pectin, scleroglucan, dextran, amylose, amylopectin, dextrin, acacia, karaya, guar, a swellable mixture of agar and carboxymethyl cellulose, a swellable composition comprising methyl cellulose mixed with a sparingly cross-linked agar, a blend of sodium alginate and locust bean gumpolymers or zein, waxes, and hydrogenated vegetable oils.

Other suitable antioxidants other than BHT include, sodium metabisulfite; tocopherols such as α, β, δ-tocopherol esters and α.-tocopherol acetate; ascorbic acid or a pharmaceutically acceptable salt thereof; ascorbyl palmitate; alkyl gallates such as propyl gallate, Tenox PG, Tenox s-1; sulfites or a pharmaceutically acceptable salt thereof; BHA; BHT; and monothioglycerol. Resveratrol (3,5,4′-tri hydroxy-trans-stilbene).

In accordance with a preferred embodiment, the final coating composition comprises an antioxidant, for example, BHT in an amount of up to five (5) percent by weight, a film-forming and/or film enhancing agent, for example, PVP in the range from about 0.05 percent to about twenty (20) percent by weight, more preferably in the range from about 0.1 percent to about five (5) percent by weight, and yet more preferably in the range from about one (1) percent to about two (2) percent, the drug or therapeutic agent, for example, paclitaxel in a therapeutically effective dosage of up to 10 μglmm² of device surface area, for example, balloon surface area and more preferably in a range from about 2 μglmm² to about 4 μglmm² of device surface area with substantially no solvent residue. The final coating composition is the result of the liquid formulation being applied to the device and then dried until substantially no residual solvent remains.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A medical device comprising:
an expandable member having a first diameter for insertion into a vessel and a second diameter for making contact with the vessel walls; and
a non-aqueous formulation of a paclitaxel, including synthetic and semi-synthetic analogs thereof, affixed to and dried onto at least a portion of the surface of the expandable member, the dried, non-aqueous formulation comprising a paclitaxel, in a therapeutic dosage in the range of up to ten micrograms per square millimeter of expandable member surface area, an antioxidant in an amount of up to 5 percent by weight relative to the amount of paclitaxel, a film forming agent in a pharmaceutically acceptable range of between 0.05 percent to about 20 percent by weight relative to the amount of paclitaxel, and substantially no volatile, non-aqueous solvent.

2. The medical device according to claim 1, wherein the expandable member comprises a balloon.

3. The medical device according to claim 2, further comprising a stent positioned over the balloon.

4. The medical device according to claim 1, wherein the antioxidant comprises butylated hydroxyl toluene.

5. The medical device according to claim 1, wherein the film forming agent comprises polyvinyl pyrrolidone.

6. A non-aqueous formulation of a paclitaxel, including synthetic and semi synthetic analogs thereof, comprising paclitaxel in a therapeutic dosage range, an antioxidant in an amount of up to 5 percent by weight relative to the amount of paclitaxel, and a film forming agent in a pharmaceutically acceptable range of between 0.05 percent to about 20 percent by weight relative to the amount of paclitaxel.

7. The non-aqueous formulation of a paclitaxel according to claim 6, wherein the antioxidant comprises butylated hydroxyl toluene.

8. The non-aqueous formulation of a paclitaxel according to claim 6, wherein the film forming agent comprises polyvinyl pyrrolidone.

9. The non-aqueous formulation of a paclitaxel according to claim 6, further comprising volatile, non-aqueous solvent.

10. The non-aqueous formulation of a paclitaxel according to claim 9, wherein the volatile, non-aqueous solvent comprises ethanol.
